(19) United States
(12) Patent Application Publication
(10) Pub. No.: US 2004/0237133 A1
(43) Pub. Date: Nov. 25, 2004

(54) Method for transformation of mono-and di-cotyledonous plants using meristematic tissue and nodal callus from dicotyledonous plants

(76) Inventors: Stephen L. Goldman, Toledo, OH (US); Sairam V. Rudrabhatla, Toledo, OH (US)

Correspondence Address: EMCH, SCHAFFER, SCHAUB & PORCELLO CO, P O BOX 916, ONE SEAGATE SUITE 1980, TOLEDO, OH, 43697

(21) Appl. No.: 10/480,865

(22) PCT Filed: Jun. 14, 2002
(86) PCT No.: PCT/US02/18966

Related U.S. Application Data
(60) Provisional application No. 60/298,542, filed on Jun. 15, 2001.
(60) Provisional application No. 60/356,563, filed on Feb. 11, 2002.

(51) Int. Cl. A01H 1/00; C12N 15/82

(52) U.S. Cl. 800/278; 435/468

(57) ABSTRACT

This invention relates to a method for the introduction of genes encoding desirable traits into both monocotyledonous and dicotyledonous plants and to plants and parts thereof produced by growing plants using this method. The time required for the production of transgenic plants is significantly decreased, while the number of transgenic plants is significantly increased. These increases are not dependent upon the use of super-virulent Agrobacterium strains. The invention also relates to an improved technique for in vitro regeneration of mono- and di-cotyledonous plants in a suitable medium containing a novel growth regulator regime that promotes cell elongation in the production of numerous somatic embryos that are regenerable into fertile plants.

### FIELD OF INVENTION

This invention relates to a new method for the introduction of genes encoding desirable traits into both monocotyledonous and dicotyledonous plants. The time required for the production of transgenic plants is significantly decreased, while the number of transgenic plants is significantly increased. These increases are not dependent upon the use of super-virulent Agrobacterium strains. Moreover, since the transformation frequency is so high in the instant invention, the identification of transformants does not require the use of a selectable marker, thus making for a more friendly DNA transfer technology.

The invention also relates to an improved technique for in vitro regeneration of mono- and di-cotyledonous plants in a suitable medium comprising a novel regulator regime that promotes cell elongation, resulting in the production of numerous somatic embryos. The time required for the production of plants is significantly decreased, while the number of plants is significantly increased.

The invention also relates to an improved technique for in vitro regeneration and transformation of soybean plants using cotyledonary nodal callus.

### BACKGROUND OF THE INVENTION

A number of different plant transformation methods have been described that may be broadly classified into two groups: Direct DNA transfer and Agrobacterium-mediated transformation. Despite the extensive amount of time, money, and energy spent on the production of transgenic plants via direct DNA uptake and with Agrobacterium-mediated transformation many problems remain that are associated with efficient production of transgenic plants. Direct DNA Uptake Technologies

Direct DNA transfer may be accomplished by Polyethylene Glycol (PEG)-mediated DNA uptake, electroporation, and biolistics. Transgenic plants may be produced from single protoplasts. Frequencies of DNA delivery to protoplasts following PEG-mediated DNA uptake is low and has not been significantly improved since 1985 (Krens et al. 1982; Paszkowski et al., 1984; Lorz et al., 1985; Potrykus et al., 1985; Vasil, 1988; Armstrong et al., 1990; Maas and Werr, 1989; Carrer et al., 1993). At an optimum, approximately 1% stable transformed protoplasts are produced and, then, only when assisted by electroporation (Shillito et al., 1985; Shillito, 1999). Of these stably transformed protoplasts, only about 1-3% may regenerate. Moreover, regeneration of protoplasts is genotype dependent and often gives rise to regenerants that express somaclonal variants.

Transgenic plants may be produced from single protoplasts following electroporation. DNA is taken into the cell following either the stabilization of pores or pore formation itself in response to voltage applied across the surface of the cell membrane (Fromm et al., 1985, 1986). In addition to low frequency, compromised regeneration, and somaclonal variation, it remains uncertain in any particular cell culture system whether a short, high voltage, or a long, low voltage is best for the production of stable transformants (Shillito, 1999). The experimental conditions that lead to a maximum recovery of stable transformants must be done on a species-by-species and cultivar-by-cultivar basis. Thus, no standard method has been established for the electroporation method of producing transgenic plants, thus decreasing the efficiency of transgenic isolation.

Transgenic plants may be produced by particle bombardment. DNA has been delivered following particle bombardment to a variety of plant tissues that include leaves, petals, endosperm, meristems, suspension cultures, immature embryos, and immature inflorescence (Bansal et al., 1992; Denchev et al., 1997; Greisbach and Klein, 1993; Knudsen and Muller, 1991; McCabe et al., 1988; Barcelo and Loazzeri, 1995; Cassas et al., 1997). Electroporation has one advantage in that the delivery of DNA is not size restricted in principle. Nevertheless, other limitations abound. First, the tissue chosen for particle bombardment must be regenerable. Tissues such as leaf segments and shoot apexes may be transformed, but do not regenerate into transgenic plants when bombarded. Furthermore, regeneration from protoplasts derived from embryogenic suspension cultures is compromised due to the difficulties in maintaining the suspension cultures for long periods of time. While in some species, long-term maintenance of suspension culture is possible, the lines chosen are limited due to dependence on genotype. An attempt to circumvent this problem uses tissues such as immature embryos. Here callus is being generated from cells in the scutellum. This route is likewise often low in efficiency and is also genotype dependent. Moreover, particle bombardment characteristically leads to high copy number insertion and to genome rearrangements. Attempts to bombard the meristem directly and thus bypass tissue culture altogether are also limited by low frequency recovery. (McCabe, 1988).

Agrobacterium-Mediated Transformation

Virulent strains of the soil bacterium A. tumefaciens introduced into wounded dicotyledonous plant tissue cause crown gall (Nester et al., 1984). The inciting agent has been shown to be a high molecular weight tumor inducing plasmid (Ti), a section of which, the T-DNA, integrates into the genome of the host plant (Chilton et al., 1980; Thomashow et al., 1997; Yadav et al., 1980). The portion of the plasmid delivered to the recipient plant maps between and includes two 23 base pair direct repeats that are designated right border (RB) and left border (LB). Linked to T-DNA are genes that, among others, encode enzymes for the production of plant hormones that stimulate cell division and cell elongation and whose expression are responsible for the plant tumor production that follows infection. Since the native genetic information between the two borders is not essential to the bacteria's survival, either a single gene or multiple genes may replace it. The loss of the native T-DNA leads to the production of a disarmed vector no longer capable of eliciting a tumorigenic response. The delivery of T-DNA follows the activation of the Ti plasmid's Vir regulon that acts in concert with a number of virulence determining chromosomal genes (Gartland, 1995).

In 1976, De Cleene and De Ley reported a list of purported natural host plants for Agrobacterium. This list contained 596 dicotyledonous and 5 monocotyledonous plants. As a result of investigations too numerous to enumerate, the host range of Agrobacterium has been shown to be far wider than originally reported (Hooykaas et al., 1984; Graves and Goldman, 1986, 1987,). In fact, Agrobacterium-mediated transformation has modified field crops and has led to the production of value-added oilseed rape (Fry J. et al. 1987), and cotton (Umbeck et al. 1987). Engineering of maize (Graves and Goldman, 1986, Fromm et al., 1990, Gordon Kamm et al. 1990), rice (Christou, 1991) and soybean (Christou et al., 1991) demonstrate different examples of different methods for introduction of foreign DNA into crop plants.

Transgenic plants may be obtained following Agrobacterium-mediated transformation of the shoot apex. This method is characterized by a low frequency of transgenic production and by the formation of chimeras. (Gould, et al., 1991). Transgenic plants may also be obtained following Agrobacterium-mediated transformation of immature embryos. Immature embryos have been the choicest explant to date since there is usually a very high frequency of callus induction and plant regeneration from immature embryos.

Following transformation, immature embryos, regardless of the method of DNA delivery, are very hard to regenerate into fertile plants (Cheng et al., 1997). In addition, it is usually very difficult to obtain immature embryos throughout the year, and their suitable stage for culture is also strictly time dependent, thus limiting their use (Oezen et al., 1998). While the frequency of transformation with immature embryos ranges from about 0.14 to about 4.3%, the number of transgenics recovered is a small fraction of the number of embryos transformed (Cheng, 1997).

The Bowen U.S. Pat. No. 5,736,369 describes transforming only monocotyledonous plants by bombarding immature embryos at the early, pro-embryo, mid-embryo, late-embryo, transitional, or early coleoptile stages of development of meristem tissue or cells that contribute to the germine cells of the meristem.

Smith et al. U.S. Pat. No. 5,164,310 describes germination of seeds for a week, excision of the shoot apex and culturing on MS+BAP medium for two days, and then inoculation with an Agrobacterium suspension. Plates were left in the hood until the inoculum was dried. The shoot apex was transferred to MS+BAP, which resulted in direct organogenesis, mode of shoot/root development.

Zhong et al. U.S. Pat. No. 5,320,961 describes a method for asexual in vitro propagation of fertile corn plants using shoot tip or apices plant tissue from a germinated seed.

Grimsley et al. U.S. Pat. Nos. 6,037,526 and 5,569,597 and related EP 0267159 relate to the transfer of viral DNA using an Agrobacterium vector to monocots.

The PCT WO 99/41975 describes isolation of apical shoot tips from young seedling, chilling of the isolated shoot tips followed by dissecting the shoot tip lengthwise. A transforming agent carrying the gene was introduced in the plant and the plantlets were regenerated from the transformed shoot tips.

The PCT WO 98/56932 describes the Agrobacterium mediated transformation of shoots, shoot tips or apexes, rooted shoots or seedlings where the transformation was carried out in an oxygenated liquid culture medium. The plant was submerged in the liquid culture medium before and after transformation, which resulted in direct organogenesis in the liquid aerated medium.

The GB 2211204 B describes preparing a mass of shoot primordium, dividing the mass into small pieces and co-culturing with Agrobacterium, which resulted in regeneration of new plants via organogenesis.

The PCT WO 99/15003 describes culturing a meristematic tissue on a high cytokinin medium to produce adventitious meristematic cells, which were proliferated in a medium containing cytokinin, copper and no growth regulators such as auxin. The meristem proliferation medium contained maltose and direct organogenesis occurred from the meristematic cells.

The Hansen U.S. Pat. No. 6,162,965 describes transformation of plants using conditions capable of inhibiting Agrobacterium-induced necrosis.

The Dong et al. U. S. Pat. No. 6,037,533 describes methods of Agrobacterium-mediated transformation of monocots.

The Guiltinan et al. U.S. Pat. No. 6,150,587 describes a method of transforming cacao tissues with Agrobacterium vectors and regenerating transgenic plants.

The PCT WO 00/42207 describes an Agrobacterium—mediated gene delivery method to individual cells in a germinated soybean meristem. The method does not involve callus—phase tissue culture, but rather involves direct induction of cells to form shoots that give rise to transgenic plants.

The PCT WO 01/77353 describes transgenic plants expressing a ragweed pollen allergen using particle bombardment or protoplast transformation using Agrobacterium-mediated transformation of leaf cells or direct gene transformation.

The Hiatt et al. U.S. Pat. No. 6,303,341 describes a method for producing immunoglobulins containing protection proteins in plants and their use.

The Agrobacterium mediated transformation website, http://www.cambiaip.org/cambiaIP/Home/welcome.htm, 2001, Ron-Rodriquez and Nottengurg, is a white paper discussing the basic scientific aspects of transformation and intellectual property aspects of methods and materials used in transformation.

The D'Halluin U.S. Pat. No. 6,140,553 describes transforming monocotyledonous plants by incubating a type I callus of corn on a MS medium supplemented with a phenolic compound prior to contacting the type I callus with a DNA fragment. D'Halluin reported an increase of T-DNA transfer to type I callus from 0.3 to 2.0% when the type I callus was pretreated in the presence of a plant phenolic compound. Type I maize callus must be subcultured before it can be efficiently used, thus extending the time needed to recover regenerated transformed plants. Using this procedure D'Halluin reports one (1) transgenic plant per hundred (100) pieces of transformed type I callus. Also, D'Halluin specifically eliminated plant growth regulators known to stimulate cell elongation.

One of the co-inventors herein, Stephen L. Goldman, has described the process for transforming plants using Agrobacterium-mediated transformation in the following U.S. Patents. Chee et al. U.S. Pat. No. 5,376,543; Chee et al. U.S. Pat. No. 5,169,770; Goldman et al. U.S. Pat. Nos. 6,020,539; 5,187,073 and 5,177,010. Inoculation of the tissues surrounding and including the apical meristem can lead to chimeric tassel development.

### SUMMARY AND OBJECTS OF THE INVENTION

The instant invention relates to a rapid, dependable, high frequency regeneration method which is universal to both monocotyledonous and dicotyledonous species and is independent of whether or not the meristem of the monocots and/or dicots are transgenic. In one of its aspects, the present invention provides a process for integrating a DNA fragment into the genome of either monocotyledonous or dicotyledonous plants.

Transgenic plants are produced more quickly and in greater number following Agrobacterium-mediated transformation of either the complete shoot and/or root meristem that has been cultured on a tissue culture medium using a plant regulator that promotes cell elongation. It is important to distinguish between the numbers of explants per treatment that are transformed from versus the number of cells per explant per treatment to which DNA is transferred. For example, it is now surprisingly found that T-DNA delivery (delivery of at least one or more genes of interest) to the shoot/root meristem is high and in excess of 90% of all shoot and/or root meristems treated, based on the expression of the GUS or GFP genes in the transformed cells or tissues. This rate of T-DNA delivery is a tremendous improvement over the prior art. In contrast, the prior T-DNA delivery methods to, for example, immature embryos, immature inflorescences and/or the shoot apex, have a rate of T-DNA delivery that approximates 10-50%, depending on the explants.

The number of cells transformed per explant is high and routine and measures 100% as can be seen in FIG. 1, 2, 3. Specifically, the expression of GUS/GFP is uniform across the surface of the explant. In contrast, following biolistics treatment the number of cells transformed is small as evidenced by the scattered isolation of transgenic sectors.

Thus, according to the present invention, the frequency of engineered cells increases significantly. Further, unlike the DNA biolistic method, the Agrobacterium-mediated transformation method of the present invention produces a high transfer rate and the majority of the transformed cells contain single copy genes, thus eliminating problems due to gene silencing.

By excising the shoot and/or root meristems and culturing the shoot and/or root meristems first in a tissue culture medium that contains plant growth regulators that promote cell elongation, the formation of regenerable callus occurs four to six weeks earlier than previously reported for type II callus. Moreover, the number of regenerable plants, as represented by the number of somatic embryos formed, increases by more than 100 fold.

In addition, by using the culturing method of the instant invention, any shoot and/or root meristem may be regenerated into plants using either somatic embryogenesis or organogenesis. Due to the increased delivery of T-DNA to the cells of the shoot/root meristems greater numbers of transformed plants can likewise be produced at increased frequencies using organogenesis as the route of regeneration. Organogenesis is callus independent and therefore requires no dedifferentiation. Dedifferentiation is known to mobilize transposable elements.

The tissue culture method of the present invention is especially useful for the efficient production of both transformed and untransformed plants such as di-haploids, virus-free cultures of ornamentals, vegetables, fruits, two-line and three-line hybrids, and for the year round cultivation of wild species of economically important plants.

The method of the present invention is also especially useful for the introduction of value added traits to apomictic plants. Apomictic plants arise from seed when the embryo has developed in the absence of fertilization. Thus, in transformed apomictics, the developed gene is stabilized immediately through maternal inheritance. The method of present invention also has other uses for apomictic plants. Thus, the present invention is also useful for the introduction of value added traits to apomictic plants following protoplast fusion between a plant carrying a gene encoding a value added trait and the apomictic; for example, a cell fusion between Sorghum and an apomictic Tripsacum. A particular benefit of this protoplast fusion is to recover novel apomictic grasses such as Sorghum in an F₂ population that segregates among other things diploid and apomictic Sorghum that express striga. Ornamental plants can also benefit. For example, due to extensive crop loss to virus infection, virus-free Gladiolus are produced. This invention allows for increased production of virus-free Gladiolus. Similar strategies are also useful with citrus crops. Thus, the invention is especially useful for integrating a DNA fragment into the genome of either monocotyledorious or dicotyledonous plants.

The method of the present invention has also been proven to be effective for immature inflorescences of corn, thus showing that any explant is capable of being transformed and regenerated at higher frequencies than previously observed.

The method of the present invention also provides that more than one T-DNA construct linked to a single T-DNA is capable of being integrated into the plant cell by cotransformation. Each T-DNA construct carries a different selectable marker such that cotransformed plant cells are distinguishable from cells that are not cotransformed.

In particular, the present invention involves the method where, prior to being contacted with the DNA fragment, a sterilized seed that produces a complete shoot meristem is germinated on a tissue culture medium containing a growth regulator that induces cell elongation for four to five days. Preferably, the growth regulator comprises at least one auxin that stimulates cell elongation in both monocots and dicots.

The germinating seed produces shoot and/or root tissue, each of which comprises a complete meristem. The complete meristem tissue within the shoot or at the root apex is cultured on the growth regulator medium. The shoot tissue contains the competent cells of the entire shoot meristem together with the leaf primordia. The root apex contains the entire root apical meristem together with the root cap.

The shoot and/or root meristem is incubated on the tissue culture medium containing a growth regulator such as auxin. The shoot/root meristem is infected with Agrobacterium containing at least one gene of interest, described in detail below. The Agrobacterium has been incubated in the presence of at least one phenolic compound such as acetosyringone. The phenolic compound is used to induce the Vir complex that, in turn, results in the increased T-DNA transfer of the gene of interest. Thereafter, the shoot and/or root meristem cell or tissue is regenerated in a suitable regeneration culture medium and cultured to grow a transformed plant.

In certain aspects, the present invention relates to a plant, or parts thereof, produced by growing the seed of the transformed plants formed by the method of the present invention, pollen of the plant, and ovule of the plant.

The present invention also relates to a plant, or parts thereof, having all the physiological and morphological characteristics of the transformed plants formed by the method of the present invention.

The present invention also relates to tissue culture of regenerable cells of a plant, wherein the tissue culture regenerates plants having all the morphological and physiological characteristics of the transformed plants. The tissue culture can be the cells being derived from a member of the group comprising leaves, pollen, embryos, meristematic cells, roots, root tips, anthers, flowers, seeds, stems, immature inflorescence, cotyledonary nodes, callus derived from cotyledonary nodes and pods.

The present invention also relates to a plant generated from the tissue culture, having all the morphological and physiological characteristics of the transformed plant.

The present invention also relates to a method for producing a seed comprising crossing a first parent plant with a second parent plant and harvesting the results in hybrid seed wherein the first parent plant or second parent plant is the transformed plant and to the hybrid seed produced by the method.

The present invention also relates to a hybrid plant, or parts thereof, produced by growing the hybrid seed and to the seed produced from the hybrid plant.

The present invention also relates to a method for producing a hybrid seed comprising crossing the transformed plant with another, different plant and to the hybrid seed produced by the method.

The present invention also relates to a hybrid plant, or parts thereof, produced by growing the hybrid seed and to the seed produced from the hybrid plant.

The present invention also relates to a plant, or parts thereof, derived from the plant, or parts thereof, by transformation with genetic material containing one or more transgenes operatively linked to one or more regulatory elements.

The present invention also relates to a method for producing a plant that contains, in its genetic material, one or more transgenes, comprising crossing a transformed plant, with either a second plant of another line of the plant or a nontransformed plant so that the genetic material of the progeny that result from the cross contains the transgene(s) operatively linked to a regulatory element, and to a plant produced by the such method.

The present invention also relates to a plant derived from the transformed plant by a single gene conversion.

The present invention also relates to a plant where the gene is selected from the group consisting a transgene, a dominant gene and a recessive gene.

### BRIEF DESCRIPTION OF THE FIGURES

The file of this patent application contains at least one drawing executed in color. Copies of this patent with color drawings will be provided by the Patent and Trademark Office upon request and payment of the necessary fee.

The Figures demonstrate the consistent protocol used in the instant invention, showing that T-DNA insertion is independent of whether the shoot and/or root meristem is from a monocotyledonous or dicotyledonous plant.

FIGS. 1A and 1B are schematic diagrams showing the genetic transformation protocol for monocots and dicots, showing both organogenesis and embryogenesis.

FIGS. 2A-2L are photographs showing regeneration and transformation of Tripsacum shoot meristems (Eastern gamagrass):

FIG. 2A shows a 3-4 day old germinated seedling;

FIG. 2B shows a callus induction from shoot meristems (5 days);

FIG. 2C & D shows embryogenic callus;

FIG. 2E shows transformed Tripsacum meristem expressing GUS activity after 20 days;

FIG. 2F shows in vitro callus and emerging plantlets expressing GFP;

FIG. 2G shows control plantlets not expressing GFP;

FIGS. 2H & I shows in vitro regenerated plants via organogenesis;

FIG. 2J shows Tripsacum transformed plants in greenhouse;

FIG. 2K shows T₀ leaves from greenhouse grown plants, positive GFP (top), negative GFP control (bottom);

FIG. 2L shows PCR analysis of primary Tripsacum.

FIGS. 3A-3L are photographs showing in vitro regeneration and transformation of corn Zea mays shoot meristems:

FIG. 3A shows 3-4 day old germinating seedling;

FIG. 3B shows callus induction from the shoot meristem;

FIG. 3C shows transformed meristem callus expressing GUS;

FIG. 3D shows embryogenic callus expressing GFP;

FIG. 3E shows globular/heart shaped embryos;

FIG. 3F shows in vitro regenerated corn plantlets;

FIG. 3G shows transgenic plants growing in the greenhouse;

FIG. 3H shows leaf tissue from greenhouse grown plants expressing GFP (right) and control tissue (left);

FIG. 3I shows greenhouse grown anther expressing GFP (left) and control anther (right);

FIG. 3J shows pollen expressing GFP;

FIG. 3K shows pollen control;

FIG. 3L shows transgenic corn seed produced from greenhouse grown plants;

FIGS. 4A-4J are photographs showing in vitro regeneration and transformation of soybean plants:

FIG. 4A shows callus initiating from the cotyledonary node;

FIG. 4B show shoot regeneration from callus derived from cotyledonary node;

FIG. 4C shows magnified view of shoot initiation;

FIG. 4D shows cotyledonary nodal callus expressing GFP;

FIG. 4E shows callus expressing GUS (top) and control (bottom);

FIG. 4F shows regenerated plantlet;

FIG. 4G shows regenerated plants in the greenhouse;

FIG. 4H shows seeds produced from transgenic plants;

FIG. 4I shows GFP expression in the leaves from plants;

FIG. 4J shows control GFP leaf.

FIG. 5 shows the PCR analysis of transformed soybean, corn and Tripsacum plants with Agrobacterium strain LBA 4404; P, Positive control; N, negative control; S1, S2-Soybean; C1, C2-Corn;T1,T2-Tripsacum; L, Ladder.

FIGS. 6A-C are photographs showing regeneration and transformation of cotton plants:

FIG. 6A shows cotton callus expressing GFP;

FIG. 6B shows regenerated cotton leaf expressing GFP;

FIG. 6C shows regenerated plantlets from transformed calli.

FIGS. 7A-C are photographs showing regeneration and transformation of wheat plants:

FIG. 7A shows wheat callus expressing GFP;

FIG. 7B shows wheat callus expressing GUS;

FIG. 7C shows regenerated wheat plantlets from transformed calli.

FIG. 8 shows Southern hybridization analysis of T₀ corn callus transformed with pBI121. DNA were digested with SacI (Lanes 5-6), XbaI (lanes 7-8), SacI and XbaI (lanes 9-12). Lanes (1-2) designated 10 pg and 50 pg of 1.9-kb SacI/XbaI fragment from pPBI121, representing one copy and five copies per diploid genome. Lanes (3-4) represent undigested corn genomic DNA. Lane 13 represents untransformed callus as negative control. Molecular size markers are indicated as L.

### DEFINITIONS

In order to provide a clear and consistent understanding of the specification and claims, including the scope to be given to such terms, the following definitions are provided.

“Untransformed cells” as used herein refers to cells, which have not been contacted with a particular DNA fragment, which will be used when applying the method of the invention. Such cells may also be derived from a transgenic plant or plant tissue, previously transformed with a different or similar DNA fragment.

The “efficiency of transformation” or “frequency of transformation” as used herein can be measured by the number of transformed cells (or transgenic organisms grown from individual transformed cells) that are recovered under the methods described herein. For example, more than 90% of the complete shoot meristems express the inserted DNA following Agrobacterium-mediated transformation under the instant invention. Nearly 100% of these transformed shoots have expressed either GUS and/or GFP. 100% of these shoot meristems produce callus containing somatic embryos too numerous to count. Each transgenic somatic embryo produces three to five shoots and three to five transgenic plants are produced per somatic embryo. For example, in the case of corn as many as eight shoots have been produce from a single callus piece.

A “transgenic plant” as used herein contains cells that replicate the delivered gene, and pass the received gene to each daughter cell in each generation and to the progeny of the next. As a result, the delivered gene is integrated in the DNA and passes from one generation to the next. The delivered gene(s) include DNA from a wide range of plant, animal, fungal, bacterial, viral, and protists sources, as well as DNA homologous to the recipient plant. The T-DNA can include selectable and/or screenable markers. However, a delivered gene need not be linked to a selectable marker. In this case, the transgenic cells can be identified following co-transformation using two separate Agrobacterium plasmids. Transgenic plants express at least one additional homologous, foreign or plant optimized gene. Transgenic plants may be produced using the method of the present invention by the Agrobacterium-mediated transformation and subsequent regeneration of the plant from the transformed cells.

“Plant growth regulators” as used herein refer to those compounds that promote cell elongation. “Plant hormones” as used herein are those hormones that promote root induction, cell division and cell elongation that lead to the formation of shoots and roots.

While not wishing to be bound to the following theory, it is thought that the beneficial effect on the transformation efficiency by incubation of the shoot and/or root meristem on a medium containing a plant regulator, i.e., auxin, is largely due to the induction of cell elongation in the particular explant. This treatment increases competence in the explant, which results in increased in DNA uptake and integration.

“Plant phenolic compounds” or “plant phenolics” as used herein invention are those molecules, which are capable of inducing a positive chemotactic response, particularly those which are capable of inducing increased Vir gene expression in Agrobacterium sp., particularly in Agrobacterium tumefaciens strains carrying T-DNA. Methods to measure chemotactic responses toward plant phenolic compounds have been described by Ashby et al. (1988) and methods to measure induction of Vir gene expression are also well known (Stachel et al., 1985; Bolton et al. 1986).

Preferred plant phenolic compounds useful with the present invention are those found in wound exudates of plant cells. One of the best-known plant phenolic compounds is acetosyringone, which is present in a number of wounded and intact cells of various plants, albeit in different concentrations. However, acetosyringone (3,5-dimethoxy-4-hydroxyacetophenone) is not the only plant phenolic which can induce the expression of Vir genes. Other examples of suitable plant phenolic compounds include: alpha-hydroxy-acetosyringone, sinapinic acid (3,5 dimethoxy-4-hydroxycinnamic acid), syringic acid (4-hydroxy-3, 5 dimethoxygenzoic acid), ferulic acid (4-hydroxy-3-methoxycinnamic acid), catechol (1,2-dihydroxybenzene), p-hydroxybenzoic acid) and vanillin (3-methoxy-4hydroxybenzaldehyde). It is contemplated that these phenolic compounds can be used to replace acetosyringone with respect to Vir induction. As used herein, the mentioned molecules are referred to as plant phenolic compounds.

The “gene and “gene of interest” as used herein includes any informational hereditary unit including regulatory sequences as well as those nucleic acid sequences involved in protein expression within the cells (including both prokaryotic and eukaryotic), including chimeric DNA constructions, plant genes and plant-optimized genes.

The genetic transformation is the stable integration of at least one foreign DNA into the genome of a plant cell, and include integration of the foreign DNA into host cell nuclear DNA and/or extranuclear DNA in organelles (e.g. chloroplasts, mitochondria and the like). The plant to be transformed may be a non-transgenic plant or may be a plant that is already transgenic but comprises one or more foreign genes tat are different than the new foreign gene or genes to be introduced.

Foreign DNA is genetic material that is not indigenous to (not normally resident in the plant before transformation or is not normally present in more than one copy. However, foreign DNA may include a further copy on an indigenous gene or genetic sequence that is introduced for purposes of co-suppression. The foreign genetic material may comprise DNA from any origin including, but not limited to plants, bacteria, viruses, bacteriophages, plasmids, plastids, mammals, and synthetic DNA constructs. The DNA may be in circular or linear form and may be single-stranded or double-stranded. The DNA may be inserted into the host plant DNA in a sense or anti-sense configuration and in single-stranded or double-stranded form. All or part of the DNA inserted into the plant cell may be integrated into the genome of the host.

The foreign DNA can also be at least one structural gene which encodes a polypeptide which imparts the desired phenotype. Alternatively, the gene may be a regulatory gene which plays a role in transcriptional and/or translation control to suppress, enhance, or otherwise modify the transcription and/or expression of an endogenous gene within the plant. It will be appreciated that control of gene expression can have a direct impact on the observable plant characteristics. The structural and regulatory genes to be inserted may be obtained from depositories such as the American Type Culture Collection, Rockville, Md. 20852 as well as by isolation from other organisms typically be the screening of genomic or cDNA libraries using conventional hybridization techniques. Sequences for specific genes may be found in various computer databases including GenBank, National Institutes of Health, as well as the database maintained by the United States Patent Office.

The “plant gene” as used herein means a gene encoded by a plant.

The “plant optimized gene” as use herein means a homologous or heterologous gene designed for plant expression.

The “gene of interest” or “delivered gene” will preferably be homologous DNA, foreign DNA or cDNA.

“Stacked genes” of interest are those containing more than one gene(s) that confer value added traits or phenotypes linked to between either the right and left T-DNA border sequences or covalently linked to the right border sequence. Alternatively, stacked genes refers to a multiple of genes that have been delivered and integrated in the host DNA of the plant cell by more than one recombination event, as in the case of co-transformation. In that case, the T-DNA constructs are in independent Agrobacterium strains.

“Expression” means the transcription and stable accumulation of the mRNA and/or protein within a cell. Expression of genes involves transcription of DNA into RNA, processing of the RNA into mRNAs in eukaryotic systems, translation of mRNA into precursor and mature proteins, followed, by, or in some cases, post-translational modification. It is not necessary that the DNA integrate into the genome of the cell in order to achieve expression. This definition in no way limits expression to a particular system and is meant to include all types including cellular, transient, in vitro, in vivo, and viral expression systems in both prokaryotic and eukaryotic cells.

“Organogenesis” means a process by which shoot and roots are developed sequentially from meristematic tissue.

“Embryogenesis” is a process of differentiation that is characterized by the formation of organized structures that resemble zygotic embryos from which shoots and roots may be produced.

### DESCRIPTION OF THE PREFERRED EMBODIMENTS

The present invention provides an efficient and universally applicable method for producing both transformed and untransformed monocotyledonous and dicotyledonous plants. The present invention thus provides an improvement over existing procedures, not only for the genetic transformation of plant cells, including both monocotyledonous and dicotyledonous plant cells, but also, for the increased efficient production of cells themselves by including in the medium a plant growth regulator. In particular, in the production of transformed cells or tissue, the plant cells or plant tissues are cultivated on a culture medium containing at least one growth regulator prior to the moment at which the complete shoot meristems are cocultured with Agrobacterium.

The present invention also provides an improved method for germinating and culturing both transformed and untransformed monocotyledonous and/or dicotyledonous plants from seeds. An undifferentiated shoot and/or root meristem cell or tissue of the mono- or di-cotyledonous plant is incubated in a suitable germinating medium containing at least one suitable growth regulator that promotes cell elongation. The shoot and/or root meristems are cultured in the dark to induce callus and somatic embryo formation on a medium containing a growth regulator (such as auxin(s)).

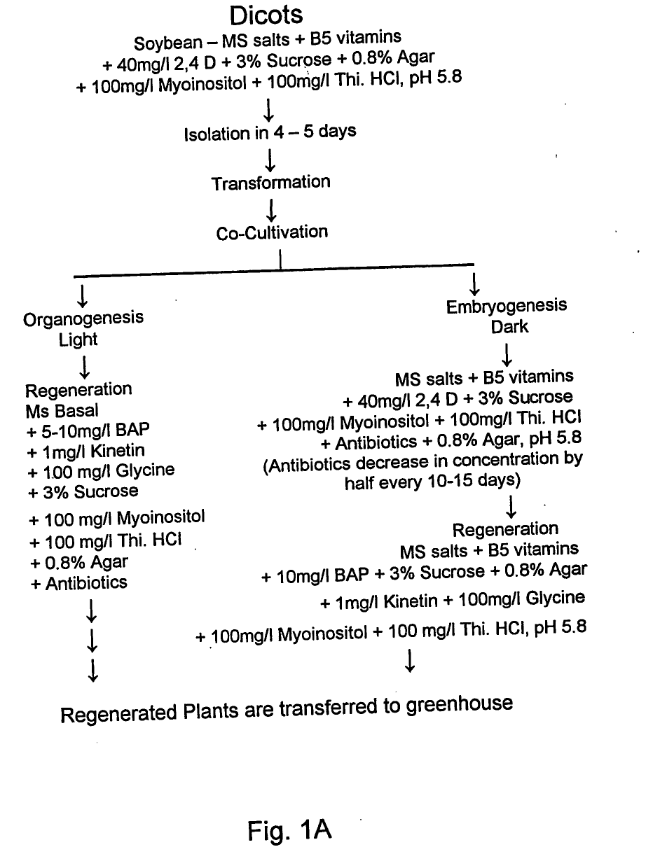

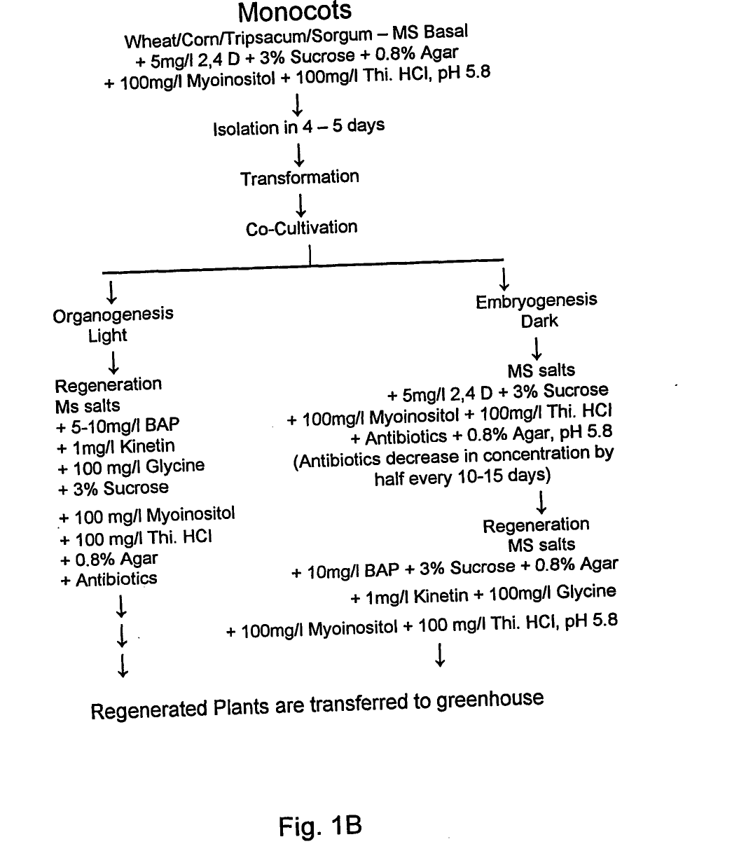

As shown in FIGS. 1A and 1B, to produce transgenic monocot and dicot plants the following steps are taken.

Prior to being contacted with the DNA fragment, a sterilized seed that produces a complete meristem is germinated on a tissue culture medium containing a growth regulator that promoted cell elongation. The shoot meristem cell or tissue is found at a shoot apex of the plant or at a root apex of the plant. The shoot meristem is located within a shoot tip, and generally appears as a dome-like structure distal to a youngest leaf primordium with at least one primordial leaf around the youngest leaf primordium. The shoot meristem typically measures less than about 3 to about 4 mm when the shoot meristem is cultured. The root apex contains the entire root apical meristem together with the root cap.

The entire shoot and/or root meristem is transferred to a callus-inducing medium containing an auxin only as the growth regulator. In one preferred embodiment, the callus containing somatic embryos is produced in the dark following incubation for 15 to 45 days. This is significant because the callus containing somatic embryos forms readily after 15 to 30 days when cereals, such as corn, wheat, Tripsacum and Sorghum are used. In addition, the calli containing somatic embryos readily form after 45 days when dicots such as soybeans are used.

In contrast, in prior art methods, the formation of callus alone derived from cereals using either immature embryos or inflorescences requires 40 to 45 days. Moreover, in the prior art methods, somatic embryos formed only after transfer to a known medium devoid of auxin. Also, known auxin-less growth medium requires an additional 25 to 45 days before somatic embryos are formed. Thus, in the preferred embodiment of the present invention, the time needed to produce either untransformed regenerated plants or transgenic regenerated plants is significantly reduced in cereals and in dicots. This reduction in time is at least a minimum two- to three-fold reduction depending upon the species.

In addition, by using the preferred embodiment, the number of somatic embryos per callus increases at least about 100 fold. Objectively, the numbers of somatic embryos per callus are too numerous to count. Potentially each transformed somatic embryos is a transgenic plant and thus, the time and labor to produce each transgenic plant is substantially reduced.

The calli and somatic embryos derived from the shoot and/or root meristems are transferred to a regeneration medium containing auxin and cytokinin. Multiple shoot induction with three to five shoots per callus are produced, which further significantly increases the number of transformed plants recovered. As many as eight shoots per callus have been produced in corn.

The entire shoot and/or root meristem explant cultivated on a tissue culture medium containing the growth regulator only, affords a flexibility for efficient production and greatly increased numbers of both untransformed and transgenic plants.

This invention also embraces both transgenic mono- and dicotyledonous plants that are capable of expressing at least one value added trait or phenotype of a gene of interest. That is, the genes may confer a beneficial trait, or phenotype, such as resistance to insects; resistance to disease, whose infectious agents include virus, bacteria, fungi and nematodes; herbicide resistance that reduce number of applications needed, thus having enormous impact on the environment; abiotic stress, which confers vigor in a challenged environment such as drought, heat, water, salt, cold and freezing; dwarfism, antibiotic resistance, expression of a pigment, expression of a fragrance, expression of a regulatory gene to control indigenous genes, plant yield enhancement and yield stability; plant improvement in nutritional quality such as grain quality which allows for the accumulation of important vitamins, amino acids, starch and storage proteins and non-starch polysaccharides, minerals; and, finally, for the production of Human Interest Products (HIPs). HIPs cover broad range of commercial and value added products that include vaccines, anti-bodies, hormones, peptides, cytokines and bioactive lipids. By using the Agrobacterium-mediated transformation method of the instant invention, great opportunities exist for the stacking of multiple genes in a single plant line, thus leading to the production of multiple value-added HIPs.

The present invention also relates to an improved organogenesis method for germinating and culturing both transformed and untransformed monocotyledonous and/or dicotyledonous plants from seeds which comprises:

i) incubating an undifferentiated shoot and/or root meristem cell or tissue of the mono- or dicot plant in a suitable germinating medium containing at least one suitable growth regulator (auxin) that promotes cell elongation, and

ii) culturing the shoot and/or root meristems in light on a medium containing at least one plant growth hormone that promotes cell division (for example, cytokinins) to induce shoot and/or root formation to form plants. This treatment induces organogenesis and therefore eliminates the dedifferentiation step known to mobilize the movement of transposable elements, and, optionally, in the case of dicots,

iii) germinating mature cotyledons of any dicot species on an auxin medium for inducing callus at the nodal region and subsequently transferring to auxin/cytokinin medium for shoot differentiation.

Gene stacking may result when more than one gene construct linked to a single T-DNA is delivered into a recipient plant cell and is integrated into the genome of the cell by co-transformation. Co-transformation removes size constraints with respect to the length of cloned fragments linked to T-DNA border (sequence). In fact, co-transformation offer a limitless opportunity to produce transgenic plants carrying novel genes from a multiple of different organisms spanning the entire living world as well as any synthetic gene constructs.

Co-transformation means that a multiple of different Agrobacterium plasmids, each carrying different T-DNA constructs are transferred and delivered to each recipient cells. Each of the different T-DNA constructs is integrated into the genome of the host plant. Preferably, each of the TNA constructs carries a different selectable marker so that cotransformed plant cell can be distinguished from those cells that are not cotransformed. The T-DNA constructs can be made in such a way that the selectable markers can be eliminated from the T-DNA construct. Marker-free transgenic plants may be generated by a variety of different means, such as described in, for example, Yoder et al. (1994) and Sugita, (1999).

Further, using the growth regulator method of the present invention, immature inflorescence have produced calli and somatic embryos on a medium containing plant growth regulators only. The time needed for the production of callus and somatic embryos is reduced 3 to 4 weeks from that previously reported. Consequently, the time needed to produce callus and somatic embryos from immature embryos is decreased also.

In one embodiment of the invention, it is preferred that the plant growth regulator, particularly auxin(s), is added to the culture medium containing the untransformed tissue and/or cells for a period of about 4 to 5 or 6 days, that is, at a time when the shoot meristem measures between about 3 to 4 mm, and preferably at least about 5 days, prior to contacting the tissues or cells with Agrobacterium containing a gene of interest covalently linked to T-DNA. In the inventors' herein experience, Tripsacum germinates mores slowly than corn and requires about 5 to 7 days before the 3-4 mm length is reached. Thus, the time required to reach the 3-4 mm length in the shoot meristem may vary from species to species. While the exact period of time in which the cultured cells are incubated in the medium containing the plant growth regulator is believed not to be critical, such incubation period preferably does not exceed about 2 weeks. Generally, it is believed that about 5 days is a useful period for the plant growth regulator to be added to the culture medium prior to the contacting untransformed tissues and/or cells with the gene of interest; or, in the case of untransformed plants, prior to regenerating the shoot and/or root meristem in a suitable regenerating medium in to a plant.

In particular, transformed mono- and dicotyledonous plant cells or tissues with one or more stacked gene constructs are produced by incubating an undifferentiated shoot meristem cell or shoot meristem tissue of the mono- or dicotyledonous plant in a suitable germinating medium containing at least one suitable plant growth regulator. The shoot meristem cell or tissue is co-cultured with a suitable phenolic primed non-supervirulent Agrobacterium, which carries any gene(s) of interest within an Agrobacterium vector. In one embodiment, the Agrobacterium includes a vir G-containing plasmid and a T-DNA containing plasmid comprising the gene of interest. Non-supervirulent as used herein refers to an Agrobacterium strain carrying a single copy of each gene comprising a single copy of gene comprising the Vir regulon. For example, LBA 4404 is a non-supervirulent strain whereas A281 is a supervirulent strain because it carries additionally copies of the Vir genes (i.e., vir B, vir C and/or vir G).

The phenolic primed non-super virulent Agrobacterium comprises a strain grown in the presence of a suitable phenolic material, which induces the Vir regulon in the vector. The T-DNA comprises at least one piece of DNA linked to the right and the left borders or to the right border sequence alone of the plasmid. The phenolic primed non-supervirulent Agrobacterium comprises a DNA molecule that delivers a gene(s) conferring a desired phenotype to the plant. Genes delivered by co-transformation require at least two separate Agrobacterium strains. It should be noted that genes linked to a single T-DNA construct in one Agrobacterium strain are formally also co-transformed when the single T-DNA construct is integrated in host DNA. The non-supervirulent Agrobacterium is pretreated with a Vir inducing phenolic compound such as acetosyringone. Suitable Agrobacterium are selected from A. tumefaciens, A. photogenes, and A. ruby.

The plant growth regulator comprises a compound that promotes cell elongation and root development, such as auxins. Preferably, the auxins comprises at least one of, but not limited to: 2,4-D (2,4-dichlorophenoxyacetic acid), decamp, IAA (indole-3-acetic acid), pictogram, NAA (-naphthalenacetic acid), IPA (indole-3-propionic acid), IBA (indole-3-butyric acid), PAA (phenyl acetic acid), BFA (benzofuran-3-acetic acid) and PBA (phenyl butyric acid).

The method of the present invention is widely useful and, in particular can be practiced where the plant is obtained from a cultivar, clone or seed. The plant can be an annual, biennial or perennial plant, and either an herbaceous or woody plant. In particular, the plants can belong to such families as the Solanaceae, Leguminosae and Gramineae. For example, the seeds can be from a monocot plant selected from barley, maize, oat, rice, wheat, rye, Sorghum, millet, Tripsacum, Triticale, forage grass and turf grass.

Still other examples of plants that can be transformed according to the method of the present invention are derived from meristems of soybean, tobacco, alfalfa, Arabidopsis, common bean and other legumes, peanut, cotton, flax, Brassica, tomato, sunflower, squash, strawberry, potato and other tubers, coffee, cocoa, pepper, Medicago sativa, lettuce, lentils, Pimpinella, anise, pine, Avena, Vigna, cucumber, poplar, spruce, clover, onion, cranberry, papaya, sugarcane, beet, wheat, barley, poppy, rape, sorghum, rose, carnation, gerbera, carrot, chicory, melon, cabbage, oat, rye, flax, walnut, citrus (including oranges, grapefruit, lemons, limes and the like), hemp, oak, rice, petunia, orchids, broccoli, cauliflower, brussel sprouts, garlic, leek, pumpkin, celery, pea, grapes, apples, pears, peaches, banana, palm, pineapple, apricot, plum, lawn grasses, maple, triticale, safflower, peanut, olive, other vegetables, other fruits and other ornamentals, dihaploids, and two-line and three-line hybrids.

In particularly preferred methods, the present invention is useful to produce such commercially important plants, such as, for example, transgenic wheat plants, maize plants, Sorghum plants, Tripsacum plants, cotton plants, soybean plants, turf grass plants, and forage grass plants.

Other aspects of the present invention relate to monocotyledonous and dicotyledonous transformed seeds and their progeny formed by the method of the present invention.

### EXAMPLES

The examples given below describe in detail the method for performing the present invention. It will be recognized that variations of this method may include different culture media or different vectors dependent upon the target plant species and the traits to be transferred into the target plants. Tripsacum, corn, wheat, cotton, Sorghum, and soybeans were chosen as the choice plants in the following examples; however, the method outlined below is universally applicable to all the mono and dicotyledonous plants without significant experimentation or modifications from the spirit and scope of this invention.

The following examples merely illustrate the nature of the invention. It will be apparent to those skilled in the art that this method can be used for any dicotyledonous plant which can be regenerated from the shoot meristem and which can be transformed by Agrobacterium. The method may also be used as is to transform monocotyledonous species without modifying the experimental parameters from the scope and spirit of the invention.

### Example 1

### In Vitro Germination Studies in Tripsacum (Eastern Gamagrass)

Sterilization of Seeds/Preparation and Culture of Explants:

Seeds of commercial variety “Pete” were obtained from a commercial grower, Shepherd Farms, Clifton Hills, Mo. Three different protocols were studied to optimize the seed germination:

a) The seeds from the commercial variety “Pete” were rinsed with detergent solution for 10-20 minutes. Then the seeds were rinsed with water for 5-6 times. Later the seeds were transferred to a sterile beaker in a laminar flow hood. The seeds were then treated with 0.1% HgCl2 for 10-15 minutes. Later they were washed with sterile water for 6-8 times and cultured on Murashige and Skoog's medium supplemented with an auxin, 2,4-dichlorophenoxyacetic acid (2-5 mg/l).

b) The seeds from the commercial variety “Pete” were rinsed with detergent solution for 10-20 minutes. Then they were rinsed with water for 5-6 times. Later the seeds were transferred to a sterile beaker in laminar flow-hood. The seeds were then treated with commercial bleach (sodium hypochlorite) at different concentrations 20, 30, 50, 100% for 15, 20, 30, 45 min respectively. Later they were washed with sterile water for 6-8 times and cultured on Murashige and Skoog's medium supplemented with an auxin, 2,4 dichlorophenoxyacetic acid (2-5 mg/l).

c) The seeds from the commercial variety “Pete” were rinsed with detergent solution for 10-20 minutes. Then they were rinsed with water for 5-6 times. Later the seeds were rinsed with 70% ethanol for 2-5 min followed by several rinses with water. Then the seeds were transferred to a sterile beaker in laminar flow-hood. The seeds were then soaked in hydrogen peroxide for 90-120 min. Later they were washed with sterile water for 6-8 times and cultured on Murashige and Skoog's medium supplemented with an auxin 2,4 dichlorophenoxy acetic acid (2-5 mg/l).

After culturing the seeds from the above three methods on auxin media for 5-10 days the shoot meristems were cultured on Murashige and Skoog's medium supplemented with an auxin 2,4 dichlorophenoxy acetic acid (5 mg/l). The above described a, b and c treatments were compared. Since the germination frequency was very high in treatment c, all the experiments were carried out using treatment c.

Results

The initial experiments included in optimizing the germination frequencies in Eastern gamagrass. Three different treatments were used. In treatment a, the germination frequency was very low. As can be seen from Table 1, in case of treatment c, the germination frequency was very high, while there was delayed germination when not using treatment c.

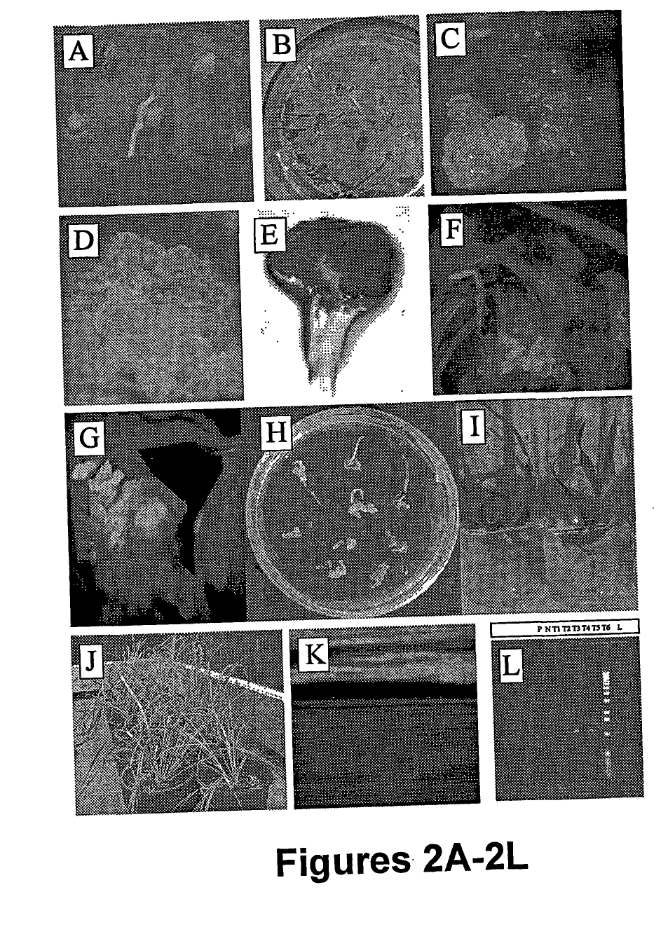

Also, FIGS. 2A-2L show photographs of the regeneration and transformation of shoot meristems from Tripsacum (Eastern gamagrass):

FIG. 2A shows a 3-4 day old germinated seedling;

FIG. 2B shows a callus induction from shoot meristems (5 days);

FIG. 2C & D shows embryogenic callus;

FIG. 2E shows transformed Tripsacum meristem expressing GUS activity after 20 days;

FIG. 2F shows in vitro callus and emerging plantlets expressing GFP;

FIG. 2G shows control plantlets not expressing GFP;

FIGS. 2H & I shows in vitro regenerated plants via organogenesis;

FIG. 2J shows Tripsacum transformed plants in greenhouse;

FIG. 2K shows T₀ leaves from greenhouse grown plants, positive GFP (top), negative GFP control (bottom);

FIG. 2L shows PCR analysis of primary Tripsacum.

TABLE 1
Comparison of germination frequencies in all three treatments

| | Treatment A | Treatment B | Treatment C |
|---|---|---|---|
| Ex # | (%)* | (%)* | (%)* |
| 1 | 20-30 | 50-60 | 65-70 |
| 2 | 18-25 | 60-65 | 70-80 |
| 3 | 15-20 | 45-50 | 75-80 |

*Means of 3 replications

### Example 2

### In Vitro Regeneration Studies in Tripsacum

Shoot meristems from Eastern gamagrass were cultured on MS medium supplemented with 100 mg/l Myo-inositol and 400 mg/l Thiamine HCl and hormone, 2,4-D at 5 mg/l. The cultures were kept in dark for induction of callus. Callus initiation was seen from day 5 after transfer to the medium. The callus induction frequencies in a range of experiments varied from 90-95% (Table 2). The mean number of shoots per callus varied from 2-5.

Calli with numerous somatic embryos were formed in 15-30 days. The calli with numerous somatic embryos were transferred on to MS medium supplemented with 100 mg/l Myo-inositol and 100 mg/l Glycine and hormones, Kinetin at 1 mg/l and BAP at 10 mg/l. The plant regeneration frequencies varied from 48 to 59% in a range of three experiments.

TABLE 2
Callus induction and Plant Regeneration frequencies in Tripsacum

| | # Shoot | # producing | Callus | No of | |
|---|---|---|---|---|---|
| | meristems | callus/ | Induction | shoots/ | Regeneration |
| Ex # | cultured | shoots* | frequency (%)* | callus* | (%)* |
| 1 | 120 | 110/65 | 91.66 | 2-3 | 59.09 |
| 2 | 150 | 135/70 | 90.00 | 2-4 | 51.85 |
| 3 | 175 | 165/80 | 94.28 | 3-5 | 48.48 |

*Means of 3 replications

### Example 3

### In Vitro Regeneration Studies in Corn

Seeds of commercial variety P15 RA 3737 from the Indiana Crop Improvement Association, Purdue were used. The seeds were rinsed with detergent solution for 10-20 minutes. Then they were rinsed with water for 5-6 times. Later the seeds were rinsed with 70% ethanol for 2-5 min followed by several rinses with water. Then the seeds were transferred to a sterile beaker in laminar flow-hood. The seeds were then soaked in 0.1% HgCl2 for 10 min. Later they were washed with sterile water for 6-8 times and cultured on Murashige and Skoog's medium supplemented with an auxin, 2,4 dichlorophenoxyacetic acid (2-5 mg/l).

After culturing the seeds from the above method on MS media for 3-5 days, the shoot meristems were cultured on Murashige and Skoog's medium supplemented with an auxin, 2,4 dichlorophenoxy acetic acid (5 mg/l).

Results

Shoot meristems from corn were cultured on MS medium supplemented with 100 mg/l Myo-inositol and 400 mg/l Thiamine HCl and hormone, 2.4-D at 5 mg/l. The cultures were kept in dark for induction of callus. Callus initiation was seen from day 5 after transfer to the medium. The callus induction frequencies in a range of experiments varied from 92-94% (Table 3). The mean number of shoots per callus varied from 4-8.

Calli with numerous somatic embryos were formed in 15-30 days. The calli with numerous somatic embryos were transferred on to MS medium supplemented with 100 mg/l Myo-inositol and 100 mg/l Glycine and hormones, Kinetin at 1 mg/l and BAP at 10 mg/l. The plant regeneration frequencies varied from 38 to 62% in all the experiments. The data shown in Table 3 is a mean of 3 replications.

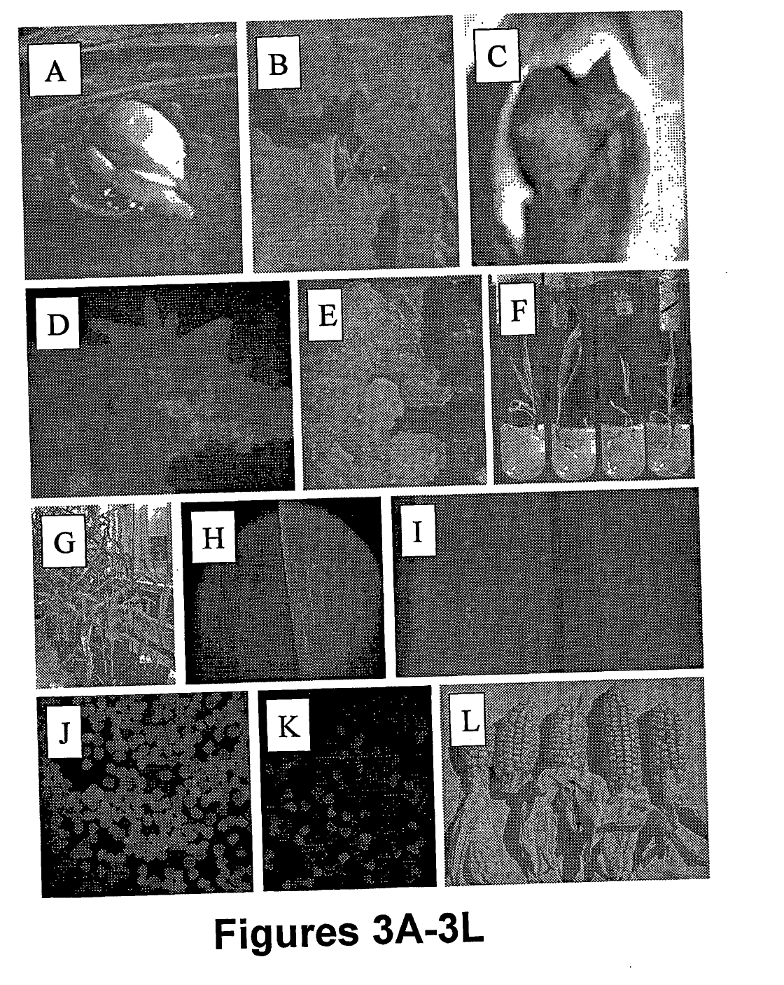

Also, FIGS. 3A-3L show photographs of in vitro regeneration and transformation of corn plants:

FIG. 3A shows 3-4 day old germinating seedling;

FIG. 3B shows callus induction from the shoot meristem;

FIG. 3C shows transformed meristem callus expressing GUS;

FIG. 3D shows embryogenic callus expressing GFP;

FIG. 3E shows globular/heart shaped embryos;

FIG. 3F shows in vitro regenerated corn plantlets;

FIG. 3G shows transgenic plants growing in the greenhouse;

FIG. 3H shows leaf tissue from greenhouse grown plants expressing GFP (right) and control tissue (left);

FIG. 3I shows greenhouse grown anther expressing GFP (left) and control anther (right);

FIG. 3J shows pollen expressing GFP;

FIG. 3K shows Pollen control;

FIG. 3L shows transgenic corn seed produced from greenhouse grown plants;

TABLE 3
Callus induction and Plant Regeneration frequencies in corn

| | | | | No of | |
|---|---|---|---|---|---|
| | # Shoot | # producing | Callus | shoots | |
| | meristems | callus/ | Induction | per | Regeneration |
| Ex | cultured | shoots* | frequency (%)* | callus * | (%)* |
| 1 | 180 | 170/65 | 94.44 | 4-5 | 38.23 |
| 2 | 165 | 155/72 | 93.93 | 4-6 | 46.45 |
| 3 | 140 | 130/80 | 92.85 | 6-8 | 61.50 |

*Means of 3 replications

### Example 4

### Somatic Embryogenesis and Genotype Independent Protocol for Corn

Shoot meristems from six different genotypes of maize were cultured in the dark for the induction of callus on modified MS medium supplemented with 100 mg l⁻¹ myo-inositol and 400 mg l⁻¹thiamine HCl in addition to 2,4-D at 5 mg l⁻¹. Callus initiation was observed on day 5 following transfer to the medium. The mean callus induction frequency in three independent experiments ranged from 86-94%, as shown in Table 4 below. The number of shoots per callus was high and varied from 3 to 6. Callus with numerous somatic embryos were formed in 10 to 15 days of incubation on auxin-supplemented medium The callus containing somatic embryos were transferred to regeneration medium [MS medium supplemented with myo-inositol and glycine, (100 mg l⁻¹), Kn (1 mg l⁻) and BAP (10 mg l⁻¹)] for maturation of somatic embryos. Green plantlets developed from the somatic embryos in 30 to 40 days following transfer to the germination medium. The plantlets were transferred to soil and hardened in the greenhouse. The plant regeneration frequency in all the six genotypes tested was reproducible and ranged from 47 to 64% thus indicating that the regeneration protocol is genotype independent. No albino or sectored plants were observed.

TABLE 4
Means for Callus induction and Plant Regeneration
Frequencies Using Shoot Meristems in Zea Mays

| | | | | | Callus | | |
|---|---|---|---|---|---|---|---|
| | | SM | Calli | Shoots | induction | shoots per | Regeneration |
| S/No | Genotype | Nos. | Nos. | Nos. | frequency | callus | (%) |
| 1 | R 23 | 161.67 | 151.67 | 72.33 | 93.74 | 5.67 | 48.73 |
| 2 | LH74 XA641 | 100.00 | 88.33 | 41.67 | 88.64 | 4.33 | 47.39 |
| 3 | LH 262 X LH252 | 61.67 | 55.67 | 28.67 | 90.00 | 4.67 | 52.44 |
| 4 | LH 198 × LH 227 | 70.00 | 60.00 | 38.33 | 86.11 | 3.67 | 64.16 |
| | LH 176 × LH 177 | | | | | | |
| 5 | DMS | 81.67 | 71.67 | 42.00 | 88.01 | 3.67 | 58.80 |
| | FR 1064 × FR 1064 | | | | | | |
| 6 | (SDMS) × LH 185 | 50.00 | 44.00 | 24.33 | 88.00 | 4.33 | 55.29 |
| Mean | | 87.50 | 78.56 | 41.22 | 89.08 | 4.39 | 54.47 |
| SE± | | 7.84*** | 6.96*** | 3.78*** | 2.48NS | 0.68NS | 6.50NS |
| CV (%) | | 15.50 | 15.30 | 15.90 | 4.80 | 26.90 | 20.70 |

Note:
***= significant at <1% (<P 0.001);
NS = Not significant

### Example 5

### Tissue Culture Studies in Soybean

Seeds of commercial variety PNP and Williams 82 were obtained from the Indiana Crop Improvement Association, Purdue. The seeds from the commercial varieties PNP and Williams 82 were rinsed with detergent solution for 10-20 minutes. Then the seeds were rinsed with water for 5-6 times. Later the seeds were rinsed with 70% ethanol for 2-5 minuets followed by several rinses with water. Then the seeds were transferred to a sterile beaker in laminar flow-hood. The seeds were then soaked in 0.1% HgCl2 for 10 min. Later they were washed with sterile water for 6-8 times and cultured on Murashige and Skoog's basal medium supplemented with B5 vitamins and an auxin 2,4 dichlorophenoxy acetic acid (5 mg/l). After culturing the seeds from the above method on auxin media for 3-5 days, the shoot meristems were cultured on cultured on Murashige and Skoog's medium supplemented with an auxin 2,4 dichlorophenoxy acetic acid (5 mg/l) for induction of callus. The callus induction frequency was 98% in both the genotypes studied (Table 5 below). The plant regeneration frequencies varied from 40-60%. The results given in the Table 5 are a means of three replications.

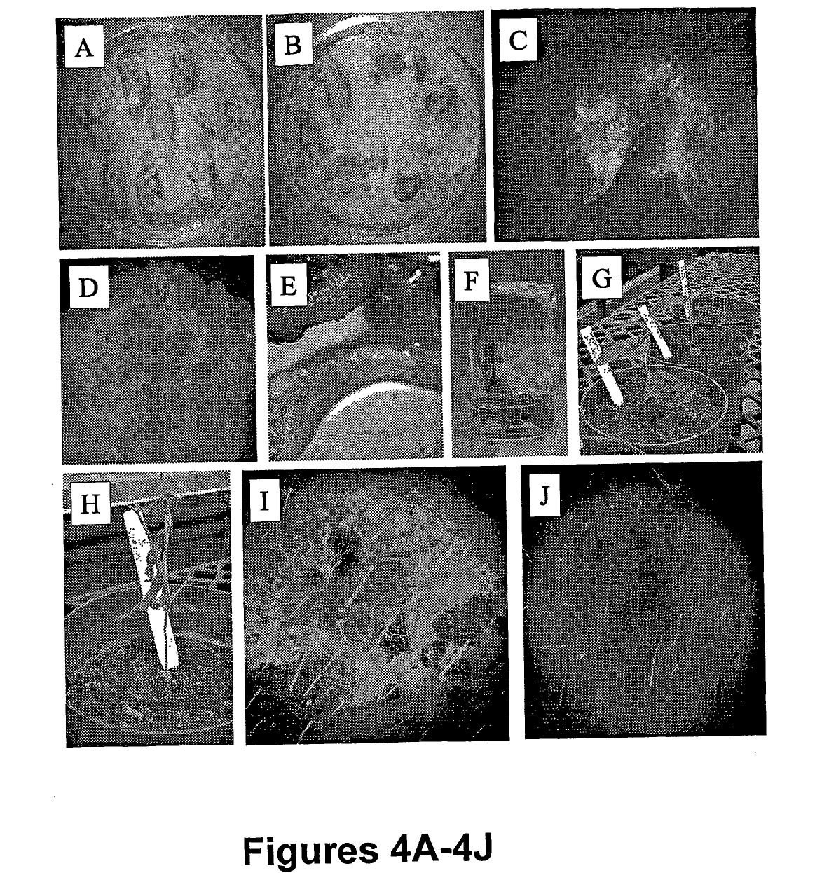

FIGS. 4A-4J are photographs showing in vitro regeneration and transformation of soybean plants;

FIG. 4A shows callus initiating from the cotyledonary node;

FIG. 4B show shoot regeneration from callus;

FIG. 4C shows magnified view of shoot initiation;

FIG. 4D shows cotyledonary nodal callus expressing GFP;

FIG. 4E shows callus expressing GUS (top) and control (bottom);

FIG. 4F shows regenerated plantlet;

FIG. 4G shows regenerated plants in the greenhouse;

FIG. 4H shows seeds produced from transgenic plants;

FIG. 4I shows GFP expression in the leaves from plants;

FIG. 4J shows control GFP leaf.

TABLE 5
Callus induction and plant regeneration frequencies in
Soybean derived from cotyledonary node

| | # Shoot | # | Callus | Plant |
|---|---|---|---|---|
| | meristems | producing | Induction | Regeneration |
| Ex. # | cultured | Calli* | (%)* | (%)* |
| 1 | 250 | 245 | 98.00 | 40.20 |
| 2 | 350 | 340 | 97.14 | 55 |
| 3 | 500 | 490 | 98.00 | 60 |

*Means of 3 replications

### Example 6

### Agrobacterium-Mediated Transformation of Tripsacum, Corn and Soybean

In the last decade, transformation technology has gained importance in the genetic manipulation of crop plants for their improvement and the study of the molecular mechanisms underlying plant gene expression and regulation. However, due to lack of highly efficient transformation procedures, the application of such biotechnological approaches has not been possible for some of the important grasses and cereals like Tripsacum, wheat, Sorghum, corn and dicots such as soybean.

a) Preparation of Agrobacterium cells, co-cultivation and regeneration of transgenic plants.

Three different strains of Agrobacterium tumefaciens viz; LBA 4404, GV 3101, EHA 105 were grown overnight on liquid LB medium (Chilton et al., 1974) supplemented with kanamycin and gentamycin (50 mg/l each) with shaking (200 rpm) at 26-28° C. The bacterial optical density was read on a spectrophotometer at 660 nm. One hour prior to the Agrobacterium reaching the desired OD, 200 uM acetosyringone was added. When the bacterial density reached 1-1.5, they were pelleted at 2000 rpm for 10 min at room temperature. After discarding the supernatant, the pellet was resuspended in resuspension medium (½ strength MS salts+Glucose+200 uM acetosyringone, pH 5.2). Cell density was readjusted after taking O.D. at 660 nm, by diluting as per requirement with resuspension medium. The shoot meristems from all species were incubated in the bacterial medium for 3 hrs and later plated on co-cultivation medium comprising A. MS medium+100 mg/l Myo inosital+100 mg/l Thiamine HCl+200 uM Acetosyringone for all cereals and grasses and B. MS Salts+B5 vitamins+100 mg/l Myo inositol+100 mg/l Thiamine HCl+200 uM Acetosyringone for dicots, in dark for 3-4 days. Later the meristems were transferred on to the callus induction media, as shown in FIG. I, with suitable antibiotics (carbencillin, 500 mg/l and cefotaxime, 250 mg/l). The cultures were regularly subcultured every 15 days decreasing the antibiotic concentrations to half each time for 4-8 weeks until the callus and somatic embryos were fully grown.

Once calli are formed they were transferred to regeneration medium and cultures were kept in light until plants were formed. The regenerated plants were transferred to soil in greenhouse.

b) Summary of transformation experiments:

At every stage after transformation the tissues were stained for GUS/GFP activity. The leaves from the primary transformants were also assayed for GUS/GFP activity. As seen in the Table 6 below, the GUS/GFP expression in case of Tripsacum ranged from 26.6-80% with all the three strains used. In case of corn, the frequency ranged from 60-86.6%. In case of Soybean, the frequencies ranged from 75-90%.

The transformation efficiency was independent of the supervirulent strain.

c) Histochemical analysis of GUS expression:

The method of Jefferson (1987) was used to assess uid A gene expression in the primary transformants at intervals between day 7 post transformation and establishment of plants in the green house and in the progeny (T1) as well. Ten to twenty seedlings were tested each time. Uninfected (controls) tissues were included in assays to detect any GUS-like activity in tissues.

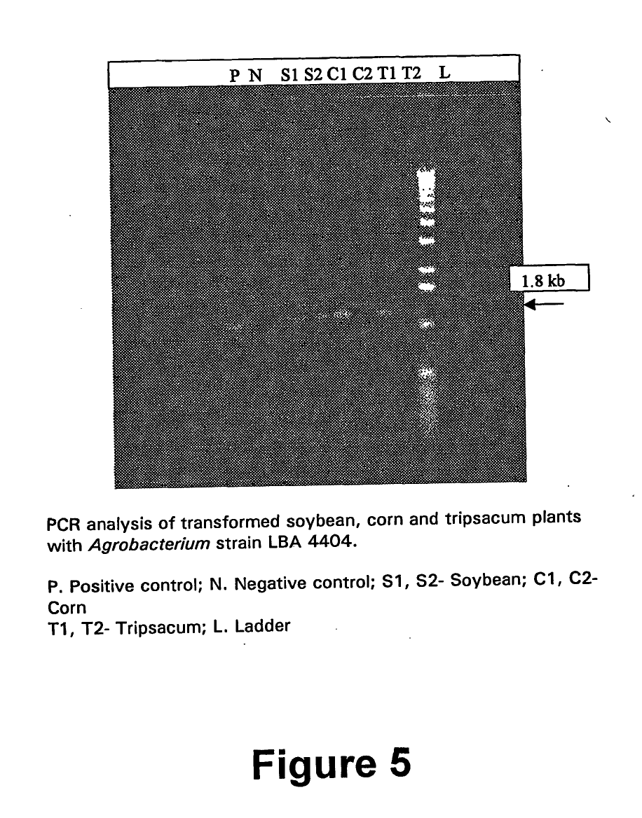

FIG. 5 shows the PCR analysis of transformed soybean, corn and tripsacum plants with Agrobacterium Strain LBA 4404.

TABLE 6
Summary of transformation experiments with
different Agrobacterium strains

| | | | # of | # of Plants | |
|---|---|---|---|---|---|
| | | | Plants | expressing | |
| | | # of | tested | GUS/GFP | GUS/GFP |
| | | shoot | for | activity | frequency |
| Ex. # | Strain | meristems | GUS/GFP | & +ve for PCR | (%) |
| Trip 1 | LBA | 60 | 10 | 8 | 80.00 |
| | 4404 | | | | |
| Trip 2 | GV3101 | 75 | 15 | 9 | 60.00 |
| Trip 3 | EHA | 65 | 15 | 6 | 40.00 |
| | 105 | | | | |
| Corn 1 | LBA | 80 | 15 | 13 | 86.66 |
| | 4404 | | | | |
| Corn 2 | GV3101 | 75 | 12 | 8 | 66.66 |
| Corn 3 | EHA | 75 | 15 | 9 | 60.00 |
| | 105 | | | | |
| Soy 1 | LBA | 120 | 25 | 20 | 80.00 |
| | 4404 | | | | |
| Soy 2 | GV3101 | 90 | 20 | 18 | 90.00 |
| Soy 3 | EHA | 85 | 20 | 15 | 75.00 |
| | 105 | | | | |

### Example 7

### Agrobacterium-Mediated T-DNA Transfer to Complete Shoots Meristems of Tripsacum

The expression of either GUS or GFP was monitored following Agrobacterium-mediated transfer to shoot meristems in calli and in leaves of the regenerated plants. FIGS. 2F and 2G show green plantlets emerging out of callus expressing GFP and a control not expressing GFP, respectively. FIG. 2E shows a typical Tripsacum shoot meristem that formed callus and was stained for GUS activity 10 d post transformation. The leaves from six primary transformants were also tested for GUS activity. All of the leaves tested on each of the plants were positive for GUS expression. In order to confirm that both T-DNA transfer and gene expression was consistently high, GFP expression was monitored in mature leaves using the green florescent microscope. For monitoring the GFP expression, the leaves were cut and placed in 100% ethanol to facilitate the chlorophyll removal. The first observation was made in 10 mins and subsequent observations were made every 10 mins until 60 mins. The expression got better each time. The results are shown in FIG. 2K.

In Table 6, the percentage of T-DNA transfer to shoot meristems is reported on a strain-by-strain basis. Transfer frequency is expressed as the percentage of GUS positive meristems that are also PCR positive. These meristems were regenerated and monitored continuously for GUS or GFP activity. LBA 4404 contains the GUS gene, which was transferred to 80% of the infected Tripsacum shoot meristems. GV 3101 carries the GFP gene, which was transferred to 60% of the infected shoot meristems. Surprisingly the super-virulent strain EHA 105 transferred T-DNA to only 40% of infected Tripsacum shoot meristems. Thus, efficient T-DNA delivery is independent of the use of super virulent strains in this system. Constructs that are vir competent but lack either the GUS or GFP genes fail to make their respective proteins following Agrobacterium-mediated transfer. As expected, these shoots neither fluoresce nor stain positive for GUS.

PCR analysis was performed with six randomly selected T₀ seedlings (FIG. 2L). Lanes T1 to T6 represents T₀ seedlings from GUS positive primary transformed plants, while lane N represents F1 seedlings from non-transformed control plants. The GUS gene used in our constructs was amplified and the size of this gene was estimated to be about 1.87 kb. All transformed seedlings showed 1.87 kb bands (lanes T1-T6). In the lanes T1 and T5 they appear to be faint, however they are distinctly seen in the original gel. The characteristic 1.87 kb band was absent for untransformed control. The PCR analysis provided additional evidence that GUS genes are present in the Tripsacum genome, and that transgene transmission is stable in the primary transformants. This observation is based on the fact that new leaves continued to express GUS activity and were also PCR positive. In no instance was GUS expression observed in absence of a positive signal.

### Example 8

### Corn Transformation

Southern Blot Analysis

The genomic DNA from corn T₀ calli were isolated using Promega GenElute™ Plant Genomic DNA kit. Southern blot and hybridization was carried out according to Sambrook et al. (2001). Genomic DNA (10 μg) was digested singly with either SacI or XbaI, and with SacI and XbaI in combination. The fragments were separated on a 1% agarose gel and transferred to a positively charged nylon membrane (Roche). A PCR-generated 1.2 kb GUS fragment was used as a probe, labeled with α[32P]-dCTP (110 TBq/mol) using a random primer DNA labeling kit (Pharmacia Biotech) according to the manufacturer's instructions. DNA marker was made using Gibco-BRL RFLP Extension Ladder System. Membranes were placed on Kodak XAR-5 film with intensifying screens at −70° C. for 3 days to visualize the hybridization results.

Results: Agrobacterium-mediated transformation of complete shoot meristem of corn

The expression of GUS gene was monitored following Agrobacterium-mediated transformation of shoot meristems, in meristem-derived callus and in leaves of the regenerated plants. FIG. 3C shows typical maize shoot meristem that formed callus and was stained for GUS activity 10 days post transformation. The leaves from the first 24 primary transformants were also tested for GUS activity. All of the leaves tested on each of the plants were positive for GUS expression. Evidence for stable integration was confirmed. In all mature leaves tested from shoots derived from this callus, positive GUS expression is always coincident with a positive PCR signal. In no instance was GUS expression observed in the absence of positive signal. In plants where transformation failed, the Southerns were negative, GUS expression was absent and there was no signal in PCR.

In Table 6, the percentage of transformed shoot meristems is reported on a strain-by-strain basis. LBA 4404 contains the GUS gene. Following infection it was successfully transferred to 86% of the shoot meristems. GV3101 carries the GFP gene. Here, T-DNA was transferred to 66% of the infected shoot meristems. Notably, the super-virulent strain EHA105 transferred T-DNA to only 60% of infected shoot meristems. Thus efficient T-DNA delivery is independent of the use of supervirulent strains using this protocol. Constructs that were Vir competent but lack either the GUS or GFP genes failed to make their respective proteins following Agrobacterium-mediated transformation. As expected these shoots neither fluoresce nor stain positive for GUS. GFP expression was constantly monitored from the 3^{rd} day post transformation until seed production. GFP expression was uniform in all somatic tissues that included the somatic embryos, regenerated plants, successive leaves, glumes and anthers (FIG. 3d,h,I,J & K). The pollen segregated 1:1 with respect to the expression of GFP fluorescence.

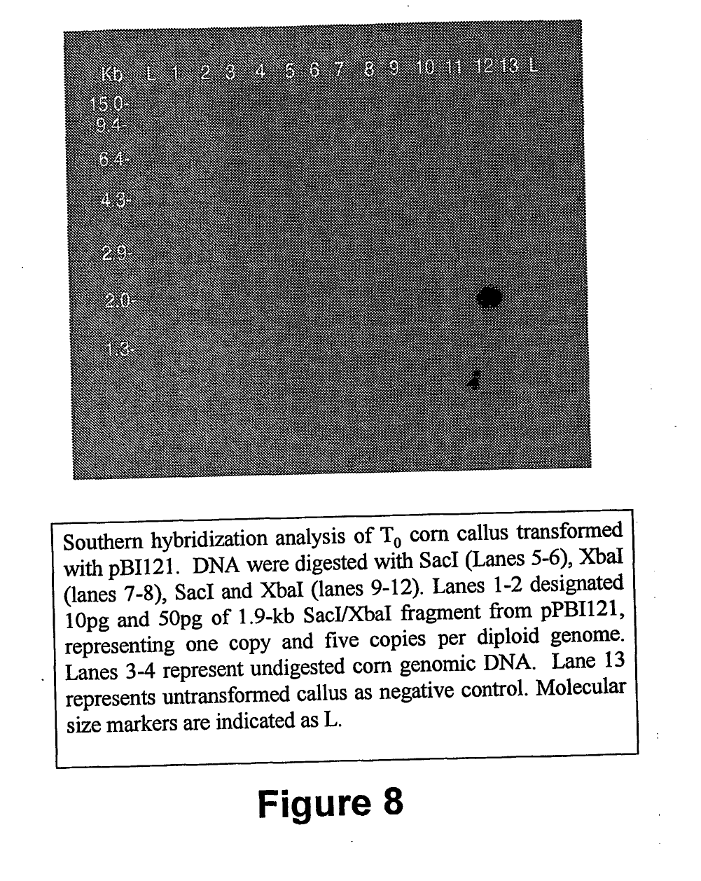

Twenty-day old corn calli (R23) on a medium-lacking antibiotic were randomly picked for Southern blot analysis, as shown in FIG. 8. From these results, it is shown that GUS incorporated into maize genomic DNA. When the GUS gene is cut with both SacI and Xba1 a single 1.9 kb fragment is generated from plasmids and 1.2 Kb PCR probe was generated. The predominant unit length hybridization signal concentrated as predicted at 1.9 kb when corn genomic DNA was double digested (lanes 9-12). Undigested genomic DNA also showed, presence of GUS gene as predicted at high molecular weights (Lanes 3-4). Lanes 7-8 show a more complex banding pattern that is consistent single digestions. DNA isolated from non-transformed plants did not hybridize with the GUS probe (Lane 11).

T₁ plants are heterozygous for the GFP gene and arose from single cell somatic embryos. As is shown in the Figures, plantlets from transgenic callus uniformly and without exception expressed GFP. Moreover, each successive mature leaf uniformly expresses GFP. Following the transition of somatic to floral meristems, it is believed the expression of GFP is uniform in tissues such as the glumes and the anthers. In contrast, GFP expression segregates in the pollen of a transgenic plant. Indeed, a 1:1 segregation was observed in these plants and is consistent with the integration of a single GFP gene in a single cell following Agrobacterium tumefaciens mediated transformation. The transfer and expression data is consistent with the high frequency of southern positives observed in unselected T₀ calli. These observations provide additional strong evidence that production of T₁ transgenic plants are frequent and stable, and of single cell origin.

### Example 9

### Effect of Optical Density (O.D.) on Transient GUS Expression in Corn Shoot Meristems

An experiment was carried out to see if O.D had any influence on the incidence of T-DNA transfer. As can be seen from Table 7, the GUS expression was very high when the O.D. of the bacterial culture was between 1-1.5. Hence, this was used as a standard for all experiments.

TABLE 7
Effect of O.D on transient expression

| | O.D at | |
|---|---|---|
| Ex # | 660 nm | GUS expression (%)* |
| 1 | 0.5 | 30-40 |
| 2 | 1 | 80-90 |
| 3 | 1.5 | 60-70 |
| 4 | 2.0 | 45-50 |
| 5 | 2.5 | 10-20 |

*Means of 3 replications

### Example 10

### Sorghum, Sorghum bicolor

Seeds of sorghum were sterilized, germinated and cultured on media as described above. Meristems were cultured on the media above. The callus initiation in Sorghum was in 6-8 days. The delay in callus initiation is due to the presence of phenols. The callus induction and plant regeneration frequencies in all the genotypes cultured varied from 60-100%.

Co-cultivation with A. tumefaciens LBA 4404 was the same as described above for corn, wheat, and Tripsacum. Following 3 days co-cultivation the shoot meristems were transferred to dark for induction of callus. The callus was transferred to light for regeneration of plants. A significant number of plants in culture were found to be GUS positive.

### Example 11

### Wheat, Triticum aestivum

Seeds of wheat were sterilized, germinated and cultured on media as described above. Meristems were cultured on the media as described in corn above. Callus initiation was seen in 5-6 days. The callus induction and plant regeneration frequencies in all the genotypes cultured varied from 40-70%.

Co-Cultivation with A. tumefaciens, LBA 4404 was the same as described above for Com, Sorghum and Tripsacum. Following 3-4 days co-cultivation the shoot meristems were transferred to dark for induction of callus. The callus was transferred to light for regeneration of plants. A significant number of plants in culture were found to be GUS positive. Similar results are observed when the GFP marker is substituted for the GUS gene.

### Example 12

### Immature Inflorescence Culture of Corn

Immature inflorescences were used to induce somatic embryogenesis using the same growth regulator regime as described above. Immature inflorescences are known to produce more morphogenic cultures since they have a number of suppressed meristematic regions that proliferate on contact with nutrient medium.

First immature inflorescences, seeds were planted in 20 cm plastic pots containing a standard greenhouse mixture of loam/sand/peat. The seedlings were grown in greenhouse conditions with supplemental lighting: 18 hours/6 hours, light/dark regime temperature regime 24° C./20° C. (day/night). With variation after 30-45 days, immature inflorescences 5-20 mm long ensheathed in several leaves were rinsed with 70% ethanol for 3-5 minutes, followed by 5 rinses in sterile distilled water. Inflorescences were extracted and cut into 2 mm segments under a sterile laminar flow hood and placed on MS medium (Murashige and Skoog, 1962) with 5 mg/l 2,4 D and cultured in the dark.

The callus initiation was seen in 8-10 days and somatic embryos were formed in 15 days. The frequency of callus induction and somatic embryogenesis varied from 60-80% in a range of genotypes.

### Example 13

### PCR Analysis of Transgenic Plants

a) Histochemical analysis of GUS expression:

The method of Jefferson (1987) was used to assess uid A gene expression in the primary transformants at intervals between day 7 post transformation and establishment of plants in the green house and in the progeny (T1) as well. Ten to twenty seedlings were tested each time. Uninfected (controls) tissues were included in assays to detect any GUS-like activity in tissues.

b) DNA analysis

DNA was isolated from calli and plants that were verified as Agrobacterium free using the procedure described by Dellaporta et al. (1983). Plasmid DNA was isolated from Agrobacterium and E.Coli following the modified method of Sambrook, Fritsch and Maniatis (1989).

Polymerase chain reaction was performed with genomic DNA from primary transformants to check for the presence of uid A gene. Two sets of primers that amplify sequences in different regions of the uid A gene were used. The PCR reactions were carried out in a total volume of 50 ul containing 1 00-500 ng template DNA, 26.96 ul milli Q water, 5 ul 10× buffer, 5 ul dNTP,s, 2 ul of primer set and 1-5 units of Taq polymerase (source). In order to amplify uid A gene sequence, PCR was initiated by a hot start at 94° C. for 4 minutes. The PCR cycles were as follows: 40 cycles which comprises 94° C. for 1 min, 50° C. for 1 min, and 72° C. for 1 min. The thermocycler used was Ericomp, twin block system. The PCR products were analyzed on a 1% agarose gel.

Results

Two samples each from soybean, corn and Tripsacum were analyzed, and all except one sample of soybean showed presence of the gene. The results can be seen in FIG. 5, which shows the PCR analysis of transformed soybean, corn and Tripsacum plants with Agrobacterium.

### Example 14

### Plant Regeneration from Soybean Cotyledonary Nodal Embryogenic Callus Induced on Auxin Medium

Mature soybean seeds were surface sterilized with 0.1% HgCl₂ for 10 mins following by five washes with sterile water and germinated on a modified MS based auxin medium containing 2 mg/l 2,4-D (FIG. 4A) for about 24 to about 96 hours. The next step was separating the two cotyledons from the hypocotyls and epicotyl regions. The cotyledon explants were collected 3-5 days post germination and incubated on a high auxin-containing medium (5-10 mg/l 2,4 D) for inducing callus at the cotyledonary nodes. Callus initiation was seen in 3-5 days (FIG. 4B). At this stage the cotyledonary explants with embryogenic callus at the nodal region were treated with Agrobacterium using the procedures described above. Alternatively, the cotyledon explants were directly incubated in Agrobacterium solution for direct organogenesis of plants. After 3 days co-cultivation the cotyledonary nodal callus explants were placed on an incubating medium comprising: high cytokinin (2-20 mg/l BAP) modified MS medium supplemented with amino acids glutamine (50 mg/l), asparagine (5-10 mg/l), cysteine (500 mg/l) and incubated in light for regenerating plants.

The explants were viewed under Olympus SZX12 epifluorescence GFP (Green Fluorescence Protein) stereomicroscope equipped with an Olympus filter cube containing 460-490 nm excitation filters and an emission filter 510 interference. The green and uniform expression seen on the cotyledons (FIG. 4D) indicates the high rate of gene delivery. Shoot regeneration started from day 5 after incubation on the medium. The number of multiple shoots per each explant was 4-5.

The frequency of callus indication was about 60 to at least about 80% and the plant regeneration frequency ranged from 40 to about 60%.

### Example 15

### Genetic Engineering of Cotton

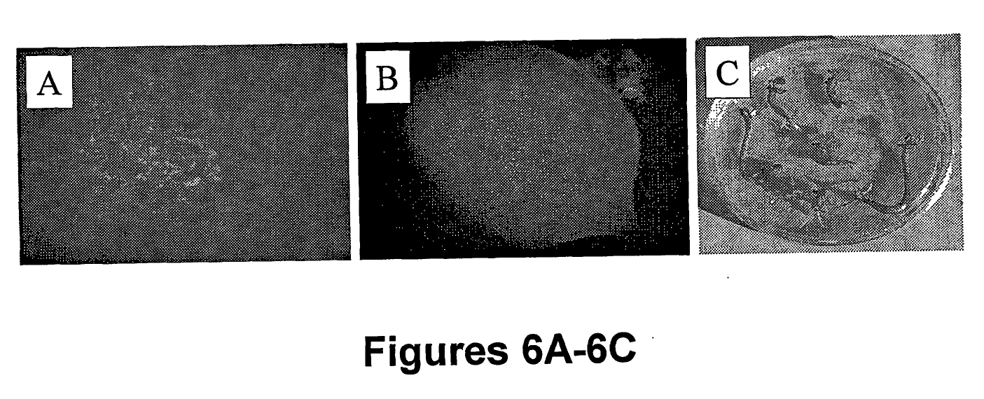

Shoot meristems from cotton were cultured on MS medium supplemented with 100 mg/l Myo-Inositol and 400 mg/l Thiamine HCl and hormone, 2,4-D at 5 mg/l. The cultures were kept in the dark for induction of callus. Callus initiation was seen from day 5 after transfer to the medium. FIGS. 6A-6C show the cotton callus expressing GFP, regenerated cotton leaf expressing GFP, and regenerated plantlets from transformed calli.

### Example 16

### Genetic Engineering of Wheat

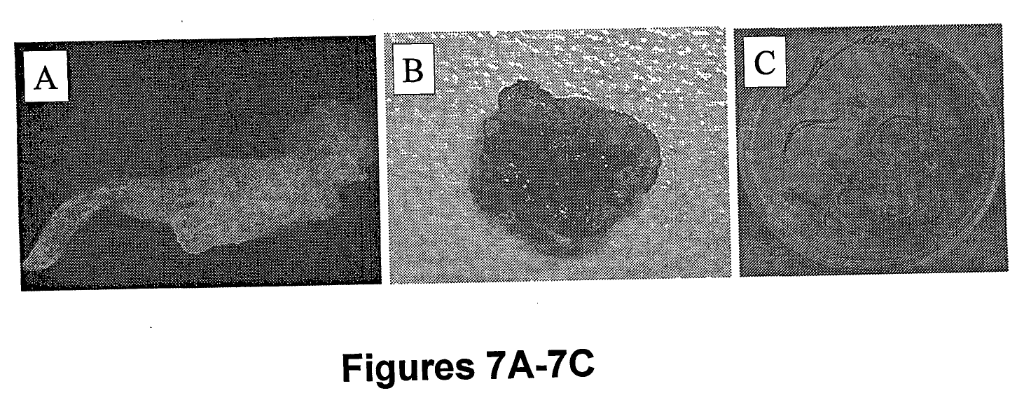

Shoot meristems from wheat were cultured on MS medium supplemented with 100 mg/l Myo-lnositol and 400 mg/l Thiamine HCl and hormone, 2,4-D at 5 mg/l. The cultures were kept in the dark for induction of callus. Callus initiation was seen from day 5 after transfer to the medium. FIGS. 7A-7C show the wheat callus expressing GFP, regenerated wheat leaf expressing GFP, and regenerated plantlets from transformed calli.

All of the compositions and methods disclosed and claimed herein can be made and executed without undue experimentation in light of the present disclosure. While the compositions and methods of this invention have been described in terms of the foregoing illustrative embodiments, it will be apparent to those skilled in the art that variations, changes, modifications, and alterations may be applied to the compositions and/or methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### References

The references discussed above and the following references, to the extent that they provide exemplary procedural or other details supplementary to those set forth herein, are specifically incorporated herein by reference.

Armstrong, C. L. et al. (1990) Plant Cell Rep. 9: 335-339.

Ashby, A. M. et al. (1988) J. Bacteriol. 170: 4181-4187.

Bansal, K. C. et al. (1992) Proc. Nat. Acad. Sci. USA 89: 3654-3658.

Barcelo, P., and Lazzeri, P. A. (1995) Meth. Mol. Biol. 49: 113-123.

Bolton, G. W. et al. (1986) Science 232: 983-984.

Carrer, H. et al. (1993) Mol. Gen. Genet. 241: 49-56.

Casas, A. M. et al. (1997) In Vitro Cell Dev. Biol. 33: 92-100.

Cheng, M. et al. (1997) Plant Physiol. 115: 971-980.

Chilton, M. D. et al. (1974) Proc. Natl. Aca. Sci. 71: 3672-3676

Chilton, M. D. et al. (1980) Proc. Natl. Aca. Sci. 77: 4060-4064.

Christou, P. (1991) Soybean Genetics Newsletter 18: 201-209.

Christou, P. et al. (1991) Bio/Technology 9: 957-962.

De Cleene, M., and De Ley, J. (1976) Bot. Rev. 42: 389-466.

Dellaporta, S. L. et al. (1983) Plant Molecular Biology Reporter 1: 19-21.

Denchev, P. D. et al. (1997) Plant Cell Rep. 16: 813-819.

Fromm, M. E. et al. (1985) Proc. Nat. Acad. Sci. USA 82: 5824-5828.

Fromm, M. E. et al. (1986) Nature: 319: 791-793.

Fromm, M. E. et al. (1990) Bio/Technology 8: 833-839.

Fry, J. et al. (1987) Plant Cell Rep. 6: 321-325.

Gordon-Kamm, W. J. et al. (1990) Plant Cell 2: 603-618.

Gould, J. et al. (1991) Plant Physiol. 95: 426-434.

Graves, A. C. F., and Goldman, S. L. (1986) Plant Mol. Biol. 7: 43-50.

Graves, A. C. F., and Goldman, S. L. (1987) J. Bacteriol. 169: 1745-1746.

Greisbach, R. J., and Klein, T. M. (1993) Lindleyana 8: 223-226.

Hooykaas-Van Slogteren, G. M. S. et al. (1984) Nature 311: 763-764.

Jefferson, R. A. (1987) Plant Molecular Biology Reporter 5: 387-405.

Knudsen, S., and Muller, M. (1991) Planta 185: 330-336.

Krens, F. A. et al. (1982) Nature 296: 72-75.

Lorz, H. et al. (1985) Mol. Gen. Genet. 199: 179-182.

Maas, C., and Werr, W. (1989) Plant Cell Rep. 8: 148-151.

McCabe, D. E. et al. (1988) Bio/Technology 6: 923-926.

Nester, E. W. et al. (1984) Anu. Rev. Plant Physiol. 35: 387-413.

Oezen, M. et al. (1998) Plant Cell, Tissue and Organ Culture 64:81-84.

Paszkowski, J. et al. (1984) EMBO. J. 3: 2717-2722.

Potrykus, I. et al. (1985) Mol. Gen. Genet. 199: 183-188.

Sambrook, J. et al. (1989) Molecular cloning. A laboratory manual, 2^{nd} edn. New York: Cold Spring Harbour Laboratory Press.

Shillito, R. D. et al. (1985) Bio/Technology 3: 1099-1103.

Shillito, R. D. (1999) in I. K. Vasil (ed). Molecular Improvement of Cereals Crops, 9-20, Kluwer Academic Publisher, Great Britian.

Stachel, S. E., et al. (1985) Nature 318: 624-628.

Sugita, K. et al. (1999) Plant Cell Reports 18: 941-947.

Thomashow, M. F. et al. (1997) Plant Journal 11: 1369-1376.

Umbeck, P. et al. (1987) Bio/technology 5: 263-266.

Vasil, V. et al. (1988) Plant Cell Rep. 7: 499-503.

Yadav, N. S. et al. (1980) Nature 287: 458-461.

Yoder, J. I., and Goldsbrough, A. P. (1994) Bio/Technology 12: 263-267.

We claim:

1. A universal method for producing transformed mono- and dicotyledonous plant cell or tissue with one or more genes of interest comprising:
a) incubating an undifferentiated shoot and/or root meristem cell or tissue of the mono- or dicotyledonous plant in a suitable germinating medium containing at least one suitable growth regulator that promotes cell elongation, and
b) infecting the shoot and/or root meristem cell or tissue with a suitable non-supervirulent Agrobacterium, containing at least one gene of interest covalently linked to T-DNA.

2. The method of claim 1, in which the Agrobacterium includes a Vir containing regulon and a T-DNA containing plasmid.

3. The method of claim 1, in which the shoot and/or root meristem cell or tissue is found at a shoot apex of the plant or at a root apex of the plant.

4. The method of claim 1, in which the shoot meristem is located within a shoot tip, the shoot meristem generally appearing as a dome-like structure distal to a youngest leaf primordium with at least one primordial leaf around the youngest leaf primordium.

5. The method of claim 4, in which the shoot meristem measures less than about 3 to about 4 mm when the shoot meristem is cultured.

6. The method of claim 3, in which the root apex contains root apical meristem together with a root cap.

7. The method of claim 1, in which the genes of interest are stacked genes comprising a multiple of T-DNA constructs that have been delivered and integrated into host DNA of the plant cell by more than one recombinant event.

8. The method of claim 7, in which the T-DNA constructs are independent Agrobacterium strains.

9. The method of claim 7, in which more than one T-DNA construct linked to a single T-DNA is integrated into the plant cell by cotransformation.

10. The method of claim 7, in which each T-DNA construct carries a different selectable marker such that cotransformed plant cells are distinguishable from cells that are not cotransformed.

11. The method of claim 10, in which the T-DNA constructs have selectable markers that are capable of being eliminated from the T-DNA construct.

12. The method of claim 1, in which immature inflorescences are produced, wherein the immature inflorescences produce calli and somatic embryos.

13. The method of claim 1, in which the rate of T-DNA delivery is at least about 90% of all shoot and/or root meristem infected.

14. The method of claim 1, further comprising the steps of:
c) regenerating the infected shoot and/or root meristem cell or tissue in a suitable regenerating culturing medium, and
d) culturing the regenerated transformed tissue to grow a transformed plant.

15. The method of claim 1, in which the Agrobacterium is grown in the presence of a suitable phenolic material.

16. The method of claim 2, in which the T-DNA comprises at least one piece of DNA linked to the right and the left borders or to the right border sequence alone of the plasmid.

17. The method of claim 1, which the growth regulator comprises at least one auxin.

18. The method of claim 17, in which the growth regulator comprises at least one of: 2,4-D (2,4-dichlorophenoxyacetic acid), dicamba, IAA (indole-3-acetic acid), picloran, NAA (-naphthalenacetic acid), IPA (indole-3-propionic acid), IBA(indole-3-butyric acid), PAA (phenyl acetic acid), BFA (benzofuran-3-acetic acid) and PBA (phenyl butyric acid).

19. The method of claim 1, in which the non-supervirulent Agrobacterium includes at least one DNA molecule that confers a desired trait to the plant.

20. The method of claim 19, in which the Agrobacterium is selected from A. tumefaciens, A. photogenes, or A. ruby.

21. The method of claim 15, in which the non-super virulent Agrobacterium is pretreated with a Vir inducing phenolic compound comprising at least one of acetosyringone, acetophenone, chalcone, or cinnamic acid.

22. The method of claim 1, in which the undifferentiated shoot and/or root meristem cell or tissue is obtained from seeds of a monocotyledonous plant.

23. The method of claim 1, in which the undifferentiated shoot and/or root meristem cell of tissue is obtained from seeds of a dicotyledonous plant.

24. The method of claim 1, in which the undifferentiated shoot and/or root meristem cell or tissue is obtained from a cultivar, clone or seed.

25. The method of claim 1, in which the plant is from an annual, biennial or perennial plant.

26. The method of claim 1, in which the plant is an herbaceous plant.

27. The method of claim 1, in which the plant is a woody plant.

28. The method of claim 1, where the shoot and/or root meristems belong to the family Solanaceae.

29. The method of claim 1, where the shoot and/or root meristems belong to the family Leguminosae.

30. The method of claim 1, where the shoot and/or root meristems belong to the family Gramineae.

31. The method of claim 1, in which the seeds are from a monocot plant selected from the group consisting of: barley, maize, oat, rice, wheat, rye, Sorghum, millet, Tripsacum, Triticale, forage grass and turf grass.

32. The method of claim 1, where plants are derived from meristems of soybean, tobacco, alfalfa, Arabidopsis, common bean and other legumes, peanut, cotton, flax, Brassica, tomato, sunflower, squash, strawberry, potato and other tubers, coffee, cocoa, pepper, Medicago sativa, lettuce, lentils, Pimpinella, anise, pine, Avena, Vigna, cucumber, poplar, spruce, clover, onion, cranberry, papaya, sugarcane, beet, wheat, barley, poppy, rape, sorghum, rose, carnation, gerbera, carrot, chicory, melon, cabbage, oat, rye, flax, walnut, oranges, grapefruit, lemons, limes and other citrus, hemp, oak, rice, petunia, orchids, broccoli, cauliflower, brussel sprouts, garlic, leek, pumpkin, celery, pea, grapes, apples, pears, peaches, banana, palm, pineapple, apricot, plum, lawn grasses, maple, triticale, safflower, peanut, olive, other vegetables, other fruits and other ornamentals, dihaploids, and two-line and three-line hybrids.

33. The method of claim 31, in which the shoot and/or root meristem cell or tissue is from a wheat plant.

34. The method of claim 31, in which the shoot and/or root meristem cell or tissue is from a maize plant.

35. The method of claim 31, in which the shoot and/or root meristem cell or tissue is from a Sorghum plant.

36. The method of claim 31, in which the shoot and/or root meristem cell or tissue is from a Tripsacum plant.

37. The method of claim 32, in which the shoot and/or root meristem cell or tissue is from a soybean plant.

38. The method of claim 31, in which the shoot and/or root meristem cell or tissue is from a turf grass plant.

39. The method of claim 31, in which the shoot and/or root meristem cell or tissue is from a forage grass plant.

40. The method of claim 32, in which the shoot and/or root meristem cell or tissue is from a cotton plant.

41. The method of claim 1, in which the genetically transformed plant comprises an apomictic plant following protoplast fusion between a plant carrying a gene encoding a value added trait and an apomictic.

42. Transformed seeds formed by the method of claim 1.

43. Transformed seeds of claim 42, comprising monocotyledonous seeds.

44. Transformed seeds of claim 42, comprising dicotyledonous seeds.

45. A product obtained from a plant transformed by the method of claim 1.

46. Progeny of the plant of claim 1.

47. The method of claim 1, in which the infected shoot or root meristems are regenerated into a plant by somatic embryogenesis.

48. The method of claim 47, further comprising culturing the infected shoot and/or root meristem in the dark to induce callus and somatic embryo formation.

49. Transformed seeds formed according to the method of claim 48.

50. The seeds of claim 49, comprising monocotyledonous seeds.

51. The seeds of claim 49, comprising dicotyledonous seeds.

52. Progeny of the plant of claim 49.

53. The method of claim 1, in which the infected shoot and/or root meristems are regenerated into a plant by organogenesis.

54. The method of claim 53, further comprising culturing the infected shoot and/or root meristems in light on a medium containing at least one plant growth hormone that promotes cell division to induce shoot and/or root formation to form transformed plants.

55. Transformed seed formed according to the method of claim 54.

56. The seeds of claim 55, comprising monocotyledonous seeds.

57. The seeds of claim 55, comprising dicotyledonous seeds.

58. Progeny of the plant of claim 55.

59. The method of claim 2, wherein the cell or tissue regenerates into plants capable of expressing at least one value added trait.

60. The method of claim 59, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

61. A universal and efficient method for producing mono- and dicotyledonous plant cell or tissue comprising: incubating an undifferentiated shoot and/or root meristem cell or tissue of the mono- or dicotyledonous plant in a suitable germinating medium containing at least one suitable growth regulator that promotes cell elongation.

62. The method of claim 61 in which the shoot and/or root meristem cell or tissue is found at a shoot apex of the plant or at a root apex of the plant.

63. The method of claim 61, in which the shoot meristem is located within a shoot tip, the shoot meristem generally appearing as a dome-like structure distal to a youngest leaf primordium with at least one primordial leaf around the youngest leaf primordium.

64. The method of claim 63, wherein the shoot meristem measures less than about 3 to about 4 mm when the shoot meristem is cultured.

65. The method of claim 62, in which the root apex contains root apical meristem together with a root cap.

66. The method of claim 61, further comprising the steps of: regenerating the infected shoot and/or root meristem cell or tissue in a suitable regenerating culturing medium, and culturing the regenerated transformed tissue to grow a plant.

67. The method of claim 61, which the growth regulator comprises at least one auxin.

68. The method of claim 67, in which the regulator comprises at least one of: 2,4-D (2,4-dichlorophenoxyacetic acid), dicamba, IAA (indole-3-acetic acid), picloran, NAA (-naphthalenacetic acid), IPA (indole-3-propionic acid), IBA (indole-3-butyric acid), PAA (phenyl acetic acid), BFA (benzofuran-3-acetic acid) and PBA (phenyl butyric acid).

69. The method of claim 61, in which the undifferentiated shoot and/or root meristem cell or tissue is obtained from seeds of a monocotyledonous plant.

70. The method of claim 61, in which the undifferentiated shoot and/or root meristem cell of tissue is obtained from seeds of a dicotyledonous plant.

71. The method of claim 61, in which the undifferentiated shoot and/or root meristem cell or tissue is obtained from a cultivar, clone or seed.

72. The method of claim 61, in which the plant is from an annual, biennial or perennial plant.

73. The method of claim 61, in which the plant is from an herbaceous plant.

74. The method of claim 61, in which the plant is from a woody plant.

75. The method of claim 61, where the shoot and/or meristems belong to the family Solanaceae.

76. The method of claim 61, where the shoot and/or meristems belong to the family Leguminosae.

77. The method of claim 61, where the shoot and/or meristems belong to the family Gramineae.

78. The method of claim 61, in which the seeds are from a monocot plant selected from the group comprising: barley, maize, oat, rice, wheat, rye, Sorghum, millet, Tripsacum, Triticale, forage grass and turf grass.

79. The method of claim 61, where plants are derived from meristems of soybean, tobacco, alfalfa, Arabidopsis, common bean and other legumes, peanut, cotton, flax, Brassica, tomato, sunflower, squash, strawberry, potato and other tubers, coffee, cocoa, pepper, Medicago sativa, lettuce, lentils, Pimpinella, anise, pine, Avena, Vigna, cucumber, poplar, spruce, clover, onion, cranberry, papaya, sugarcane, beet, wheat, barley, poppy, rape, sorghum, rose, carnation, gerbera, carrot, chicory, melon, cabbage, oat, rye, flax, walnut, oranges, grapefruit, lemons, limes and other citrus, hemp, oak, rice, petunia, orchids, broccoli, cauliflower, brussel sprouts, garlic, leek, pumpkin, celery, pea, grapes, apples, pears, peaches, banana, palm, pineapple, apricot, plum, lawn grasses, maple, triticale, safflower, peanut, olive, other vegetables, other fruits and other ornamentals, dihaploids, and two-line and three-line hybrids.

80. The method of claim 78, in which the shoot and/or root meristem cell or tissue is from a wheat plant.

81. The method of claim 78, in which the shoot and/or root meristem cell or tissue is from a maize plant.

82. The method of claim 78, in which the shoot and/or root meristem cell or tissue is from a Sorghum plant.

83. The method of claim 78, in which the shoot and/or root meristem cell or tissue is from a Tripsacum plant.

84. The method of claim 79 in which the shoot and/or root meristem cell or tissue is from a soybean plant.

85. The method of claim 78 in which the shoot and/or root meristem cell or tissue is from a turf grass plant.

86. The method of claim 78, in which the shoot and/or root meristem cell or tissue is from a forage grass plant.

87. The method of claim 79, in which the shoot and/or root meristem cell or tissue is from a cotton plant.

88. The method of claim 61, in which the plant comprises an apomictic plant following protoplast fusion between a plant carrying a gene encoding a value added trait and an apomictic.

89. Seeds formed by the method of claim 61.

90. Seeds of claim 89, comprising monocotyledonous seeds.

91. Seeds of claim 9, comprising dicotyledonous seeds.

92. A product obtained from a plant produced by the method of claim 61.

93. Progeny of the plant of claim 61.

94. The method of claim 61, in which the shoot and/or root meristems are regenerated into a plant by somatic embryogenesis.

95. The method of claim 94, further comprising culturing the shoot and/or root meristems in the dark to induce callus formation and somatic embryo formation.

96. Seeds formed according to the method of claim 95.

97. The seeds of claim 96, comprising monocotyledonous seeds.

98. The seeds of claim 96, comprising dicotyledonous seeds.

99. Progeny of the plant of claim 96.

100. The method of claim 61, in which the shoot and/or root meristems are regenerated into a plant by organogenesis.

101. The method of claim 100, further comprising culturing the shoot and/or root meristems in light on a medium containing at least one plant growth hormone that promotes cell division to induce shoot or root formation to form transformed plants.

102. Seed formed according to the method of claim 101.

103. Seeds of claim 102, comprising monocotyledonous seeds.

104. Seeds of claim 102, comprising dicotyledonous seeds.

105. Progeny of the plant of claim 102.

106. The method of claim 61, wherein the tissue regenerates into plants capable of expressing at least one value added trait.

107. The method of claim 61, wherein the value added trait comprises herbicide resistance, insect resistance, resistance to bacterial, fungal, nematode or viral disease, yield enhancement, improved nutritional quality, enhanced yield stability, or production of human interest products.

108. The method of claim 1, in which the dicotyledonous plants are incubated in the germinating medium which contains at least one auxin for a time ranging from about 24 to about 96 hours to allow development of dicotyledonous tissue.

109. The method of claim 108, further comprising the steps of:
i) separating the two cotyledons from hypocotyls and epicotyl tissue;
ii) incubating the cotyledons on an auxin medium comprising about 5 to about 10 mg/l 2,4-D (2,4-dichlorophenoxyacetic acid) for about 3 to about 5 days to allow incubation of callus at a nodal region; and
iii) inoculating the nodal callus, region with a disarmed Agrobacterium tumefaciens vector.

110. The method of claim 109, further comprising the step of:
iv) regenerating at least one plant from the nodal callus on a medium comprising at least one auxin and at least one cytokinin.

111. The method of claim 109, further comprising the step of:
iv) regenerating at least one plant from at least one cotyledonary node via organogenesis on a medium comprising at least one cytokinin.

112. Transformed seeds formed by the method of claim 108.

113. Transformed seeds formed by the method of claim 109.

114. Transformed seeds formed by the method of claim 110.

115. A product obtained from a plant transformed by the method of claim 108.

116. A product obtained from a plant transformed by the method of claim 110.

117. A product obtained from a plant transformed by the method of claim 111.

118. Progeny of the plant of claim 108.

119. Progeny of the plant of claim 110.

120. Progeny of the plant of claim 111.

121. The method of claim 108, wherein the cell or tissue regenerates into plants capable of expressing at least one value added trait.

122. The method of claim 110, wherein the cell or tissue regenerates into plants capable of expressing at least one value added trait.

123. The method of claim 111 wherein the cell or tissue regenerates into plants capable of expressing at least one value added trait.

124. The method of claim 108, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

125. The method of claim 110, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

126. The method of claim 111, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

127. The method of claim 108, in which the shoot and/or root meristem cell or tissue is from a dicotyledonous plant.

128. The method of claim 127, further comprising the steps of:
i) separating the two cotyledons from hypocotyls and epicotyl tissue; and
ii) incubating the cotyledons on an auxin medium comprising about 5 to about 10 mg/l 2,4-D (2,4-dichlorophenoxyacetic acid) for about 3 to about 5 days to allow inucation of callus at a modal region.

129. The method of claim 128, further comprising the step of:
iii) regenerating at least one plant from the nodal callus on a medium comprising at least one auxin and at least one cytokinin.

130. The method of claim 129, further comprising the step of:
iv) regenerating at least one plant from at least one cotyledonary node via organogenesis on a medium comprising at least one cytokinin.

131. Seeds formed by the method of claim 127.

132. Seeds formed by the method of claim 129.

133. Seeds formed by the method of claim 130.

134. A product obtained from a plant formed by the method of claim 127.

135. A product obtained from a plant formed by the method of claim 129.

136. A product obtained from a plant formed by the method of claim 130.

137. Progeny of the plant of claim 127.

138. Progeny of the plant of claim 129.

139. Progeny of the plant of claim 130.

140. The method of claim 127, wherein the cell or tissue regenerates into plants capable of,expressing at least one value added trait.

141. The method of claim 129, wherein the cell or tissue regenerates into plants capable of expressing at least one value added trait.

142. The method of claim 130, wherein the cell or tissue regenerates into plants capable of expressing at least one value added trait.

143. The method of claim 127, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

144. The method of claim 129, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

145. The method of claim 130, wherein the gene confers at least one value added trait comprising: herbicide resistance, insect resistance, resistance to bacterial, fungal, nematode or viral disease, enhanced yield enhancement, improved nutritional quality, enhanced yield stability, or production of human interest products; resistance to abiotic stress which confers vigor in a challenged environment including drought, heat, water, salt, cold and freezing; dwarfism; antibiotic resistance; expression of a pigment; expression of a fragrance; and, expression of a regulatory gene to control indigenous genes.

146. A plant, or parts thereof, produced by growing the seed of claim 42.

147. Pollen of the plant of claim 146.

148. An ovule of the plant of claim 147.

149. A plant, or parts thereof, having all the physiological and morphological characteristics of the plant of claim 146.

150. A tissue culture of regenerable cells of a plant, wherein the tissue culture regenerates plants having all the morphological and physiological characteristics of the plant of claim 147.

151. The tissue culture according to claim 150, the cells being derived from a member of the group comprising leaves, pollen, embryos, meristematic cells, roots, root tips, anthers, flowers, seeds, stems immature inflorescence, cotyledonary nodes, callus derived from cotyledonary nodes and pods.

152. A plant generated from the tissue culture of claim 151, having all the morphological and physiological characteristics of the plant of claim 146.

153. A method for producing a seed comprising crossing a first parent plant with a second parent plant and harvesting the resultant hybrid seed wherein the first parent plant or second parent plant is the plant of claim 146.

154. A hybrid seed produced by the method of claim 153.

155. A hybrid plant, or parts thereof, produced by growing the hybrid seed of claim 154.

156. A seed produced from the hybrid plant of claim 155.

157. A method for producing a hybrid seed comprising crossing the plant according to claim 146 with another, different plant.

158. The hybrid seed produced by the method of claim 157.

159. A hybrid plant, or parts thereof, produced by growing the hybrid seed of claim 158.

160. A seed produced from the hybrid plant of claim 159.

161. A plant, or parts thereof, derived from the plant, or parts thereof, of claim 146, by transformation with genetic material containing one or more transgenes operatively linked to one or more regulatory elements.

162. A method for producing a plant that contains, in its genetic material, one or more transgenes, comprising crossing a plant of claim 161, with either a second plant of another line of the plant or a nontransformed plant so that the genetic material of the progeny that result from the cross contains the transgene(s) operatively linked to a regulatory element.

163. A plant produced by the method of claim 162.

164. A plant derived from the plant of claim 146 by a single gene conversion.

165. A plant of claim 164, wherein the gene is selected from the group consisting a transgene, a dominant gene and a recessive gene.
